SEQUENCE LISTING (35 sequences)

SEQ ID NO: 1 - protein, 376 aa, Artificial Sequence
  TK Amino Acid Sequence
MASYPCHQHASAFDQAARSRGHSNRRTALRPRRQQEATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVTPQGAEPQSNAGPRPHIGETLFTLFRAPELLAPNGDLYNVFAWALDVLAKRLRPMHVFILDYDQSPAGCRDALLQLTSGMVQTHVTTPGSIPTICDLARTFAREMGEAH

SEQ ID NO: 2 - DNA, 1131 bp, Artificial Sequence
  TK nucleotide sequence
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 3 - protein, 10 aa, Artificial Sequence
  Linker
GGGGSGGGGS

SEQ ID NO: 4 - protein, 10 aa, Artificial Sequence
  Amino acid sequence of illustrative linker
ACGSLSCGSF

SEQ ID NO: 5 - protein, 10 aa, Artificial Sequence
  Linker
EAAAREAAAR

SEQ ID NO: 6 - protein, 20 aa, Artificial Sequence
  Linker
EAAAREAAAREAAAREAAAR

SEQ ID NO: 7 - protein, 20 aa, Artificial Sequence
  Linker
ACGSLSCGSFACGSLSCGSF

SEQ ID NO: 8 - protein, 10 aa, Artificial Sequence
  Linker
ATSATATSAT

SEQ ID NO: 9 - protein, 5 aa, Artificial Sequence
  Forward Linker
GGGGS

SEQ ID NO: 10 - DNA, 24 bp, Artificial Sequence
  Forward Primer Sequence
gtaatgacaagcgcccagataaca

SEQ ID NO: 11 - DNA, 24 bp, Artificial Sequence
  Reverse Primer Sequence
gcacgccgcgtccccggccgatat

SEQ ID NO: 12 - DNA, 29 bp, Artificial Sequence
  Forward Primer Sequence
cgtcttggccaaatgtctccgtcccatgc

SEQ ID NO: 13 - DNA, 29 bp, Artificial Sequence
  Reverse Primer Sequence
gcatgggacggagacatttggccaagacg

SEQ ID NO: 14 - DNA, 34 bp, Artificial Sequence
  Forward nTK
atatgaattcgcttcgtacccctgccatcaacac

SEQ ID NO: 15 - DNA, 34 bp, Artificial Sequence
  Reverse cTK
atatggatccgttagcctcccccatctcccgggc

SEQ ID NO: 16 - DNA, 37 bp, Artificial Sequence
  Reverse nTK152
atatctcgagtcaggcatgtgagctcccagcctcccc

SEQ ID NO: 17 - DNA, 31 bp, Artificial Sequence
  Forward cTK152
atatgctagcatgccgcccccggccctcacc

SEQ ID NO: 18 - DNA, 37 bp, Artificial Sequence
  Reverse nTK180
atatctcgagtcagcccataaggtatcgcgcggccgg

SEQ ID NO: 19 - DNA, 37 bp, Artificial Sequence
  Forward cTK180
atatgctagcatgagcatgaccccccaggccgtgctg

SEQ ID NO: 20 - DNA, 37 bp, Artificial Sequence
  Reverse nTK195
atatctcgagtcacgggatgagggccacgaacgccag

SEQ ID NO: 21 - DNA, 37 bp, Artificial Sequence
  Forward cTK195
atatgctagcatgccgaccttgcccggcacaaacatc

SEQ ID NO: 22 - DNA, 37 bp, Artificial Sequence
  Reverse nTK265
atatctcgagtcacgtccccgaaagctgtccccaatc

SEQ ID NO: 23 - DNA, 37 bp, Artificial Sequence
  Forward cTK265
atatgctagcatggccgtgccgccccagggtgccgag

SEQ ID NO: 24 - DNA, 37 bp, Artificial Sequence
  Reverse nTK300
atatctcgagtcaaactcgggggcccgaaacagggta

SEQ ID NO: 25 - DNA, 37 bp, Artificial Sequence
  Forward cTK300
atatgctagcatggctggcccccaacggcgacctgta

SEQ ID NO: 26 - DNA, 51 bp, Artificial Sequence
  Forward Linker
gatccggtggcggagggagcggtggcggagggagcggtggcggagggagcg

SEQ ID NO: 27 - DNA, 46 bp, Artificial Sequence
  Reverse Linker
gctccctccgccaccgctccctccgccaccgctccctccgccaccg

SEQ ID NO: 28 - DNA, 34 bp, Artificial Sequence
  Forward nTK
atatgctagcatggcttcgtacccctgccatcaa

SEQ ID NO: 29 - DNA, 34 bp, Artificial Sequence
  Reverse nTK
atatgaattccgtccccgaaagctgtccccaatc

SEQ ID NO: 30 - DNA, 61 bp, Artificial Sequence
  Forward Id
atatgaattcggtggcggagggagcggtggcggagggagccataaattcccacttggtctg

SEQ ID NO: 31 - DNA, 28 bp, Artificial Sequence
  Reverse Id
atatctcgagattaaccctcactaaagg

SEQ ID NO: 32 - DNA, 37 bp, Artificial Sequence
  Forward MyoD
atatgctagcatgccggagtggcagaaagttaagacg

SEQ ID NO: 33 - DNA, 61 bp, Artificial Sequence
  Reverse MyoD
atatggatccgctccctccgccaccgctccctccgccaccccgaattcgagctcgcccggg

SEQ ID NO: 34 - DNA, 37 bp, Artificial Sequence
  Forward cTK
atatggatcctgcgtgccgccccagggtgccgagccc

SEQ ID NO: 35 - DNA, 34 bp, Artificial Sequence
  Reverse cTK
atatctcgagtcagttagcctcccccatctcccg

(19) United States
(12) Patent Application Publication
(10) Pub. No.: US 2009/0075313 A1
(43) Pub. Date: Mar. 19, 2009

(54) Split protein fragments, split protein systems, methods of making split protein systems, and methods of using split protein systems

(75) Inventors: Tarik F. Massoud, Cambridge (GB); Ramasamy Paulmurugan, Mountain View, CA (US); Sanjiv Sam Gambhir, Portola Valley, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Stanford University, Palo Alto, CA (US)

(21) Appl. No.: 12/151,271

(22) Filed: May 5, 2008

Related U.S. Application Data
(60) Provisional application No. 60/927,554, filed on May 4, 2007.

(51) Int. Cl. C12Q 1/48; C12N 9/12; C12N 5/06; C12P 21/04

(52) U.S. Cl. 435/15; 435/194; 435/69.1; 435/375

(57) ABSTRACT

Split protein herpes simplex virus type 1 thymidine kinase [HSV1-TK or TK] TK fragments, split protein TK systems, methods of imaging protein-protein interactions, methods of cellular localization of proteins, methods of evaluating protein translocation and trafficking, and the like, are provided. In addition, the present disclosure includes compositions used in and methods relating to non-invasive imaging (e.g., positron emission tomography (PET) imaging) in vivo and in vitro.

### CROSS-REFERENCE TO RELATED APPLICATION

This application claims priority to U.S. provisional application entitled, “SPLIT PROTEIN FRAGMENTS, SPLIT PROTEIN SYSTEMS, METHODS OF MAKING SPLIT PROTEIN SYSTEMS, AND METHODS OF USING SPLIT PROTEIN SYSTEMS,” having Ser. No. 60/927,554, filed on May 4, 2007, which is entirely incorporated herein by reference.

### STATEMENT REGARDING FEDERALLY SPONSORED RESEARCH OR DEVELOPMENT

This invention was made with government support under Grant Nos.: ICMIC P50 CA114747 awarded by the National Cancer Institute, and RO1 CA082214 awarded by the National Cancer Institute. The government has certain rights in the invention.

### BACKGROUND

Protein-protein interactions are vital to most cellular functions, being associated with processes as diverse as enzymatic activity, signal transduction, immunological recognition, and DNA repair and replication. Although these interactions have been among the most difficult aspects of cell biology to investigate, a multitude of experimental qualitative and quantitative techniques have been developed with the common goal of understanding these ubiquitous interactions. These strategies include cell culture methods to monitor dynamic real-time protein-protein interactions in living cells, e.g., using fluorescence resonance energy transfer (FRET) microscopy. Further extension of these methods to noninvasively detect, localize, and quantify protein dimerization in the setting of an intact living experimental or clinical subject could have important implications for a wide variety of biological research endeavors, drug discovery, and molecular medicine. In particular, the visual representation, characterization, quantification, and timing of these biological processes in living subjects could create unprecedented opportunities to complement available in vitro or cell culture methodologies, in order: (i) to accelerate the evaluation in living subjects of novel drugs that promote or inhibit active homodimeric or heterodimeric protein assembly, and (ii) to characterize more fully known protein-protein interactions (e.g., the reasons for, and the factors that drive their association) in the context of whole-body physiologically-authentic environments.

Thymidine kinases are key enzymes in the pyrimidine salvage pathway catalyzing the transfer of the y-phosphate group from ATP to the 5′-OH group of thymidine in the presence of magnesium ions. In crystal structures of herpes simplex virus type 1 thymidine kinase (HSV1-TK, here abbreviated as TK) the protein is a homodimer; one asymmetric crystal unit is composed of two 376-residue subunits (FIG. 1a) (SEQ ID Nos: 1 and 2). TK enzyme phosphorylates a wide range of nucleoside analogues, allowing selective anti-herpetic and viral vector-based gene therapies.

The two main categories of substrates for TK, uracil nucleoside derivatives labeled with radioactive iodine (e.g., FIAU or radiolabeled 2′-fluoro-2′-deoxyarabinofuranosyl-5-ethyl uracil (FEAU)), and acycloguanosine derivatives labeled with radioactive ¹⁸F-Fluorine (e.g., fluoropenciclovir [FPCV] or 9-(4-[¹⁸F]-fluoro-3-hydroxymethylbutyl)-guanine [FHBG]), have been investigated in the last few years as reporter probes for imaging HSV1-tk reporter gene expression. These radiolabeled reporter probes are transported into cells, and are trapped as a result of phosphorylation by TK. When used in non-pharmacological tracer doses, these substrates can serve as PET or single photon emission computed tomography (SPECT) targeted reporter probes by their accumulation in just the cells expressing the HSV1-tk gene. Additionally, a mutant version of this gene, HSV1-sr39tk was derived using site-directed mutagenesis to obtain an enzyme (HSV1-sr39TK) more effective at phosphorylating ganciclovir/penciclovir, and also less efficient at phosphorylating thymidine, with consequent gain in imaging signal²⁵.

The principle of the protein fragment-assisted complementation assay (PCA) strategy for detecting protein-protein interactions was first demonstrated by Pelletier et al. using the enzyme dihydrofolate reductase (DHFR), following inspiration from a 1994 paper by Johnsson and Varshavsky describing what they called the ‘ubiquitin split protein sensor’. In all PCAs, splitting a specific reporter protein into two distinct fragments abolishes its function. Bringing the two fragments back together in a controlled manner then restores partial functional activity (FIG. 1b). Selected fragments of many proteins can associate to produce functional bimolecular complexes; the PCA system can therefore be generalized for a number of enzymes for detection of protein-protein interactions, examples including DHFR, glycinamide ribonucleotide (GAR) transformoylase, aminoglycoside and hygromycin B phosphotransferases (all reviewed by Michnick et al.), E. coli TEM-1 β-lactamase, green fluorescent protein (GFP) and its variants, the molecular imaging reporters Firefly luciferase and Renilla luciferase, and more recently, Gaussia luciferase and Click Beetle luciferase. Split reporter systems have been used to date in several model organisms, including E. coli, yeast, C. elegans, and mice.

### SUMMARY

Embodiments of the present disclosure provide for split protein herpes simplex virus type 1 thymidine kinase [HSV1-TK or TK] TK fragments, split protein TK systems, methods of imaging protein-protein interactions, methods of cellular localization of proteins, methods of evaluating protein translocation and trafficking, and the like. In addition, the present disclosure includes compositions used in and methods relating to non-invasive imaging (e.g., positron emission tomography (PET) imaging) in vivo and in vitro.

One exemplary split protein herpes simplex virus type 1 thymidine kinase (TK) system, among others, includes: a first TK protein including a first TK self complementing fragment, wherein the first TK self complementing fragment comprises a first portion of a TK protein, and a second TK protein including a second TK self complementing fragment, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein.

One exemplary method of producing the split protein system, among others, includes: providing a first vector that includes a first polynucleotide that encodes a first herpes simplex virus type 1 thymidine kinase (TK) protein including a first TK self complementing fragment, wherein the first TK self complementing fragment comprises a first portion of a TK protein; expressing the first polynucleotide to produce the first TK protein in a first system; providing a second vector that includes a second polynucleotide sequence that encodes a second TK protein including a second TK self complementing fragment, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein; and expressing the second polynucleotide to produce the second TK protein in a second system.

One exemplary method of detecting protein-protein interaction, among others, includes: providing a first vector that includes a first polynucleotide that encodes a first herpes simplex virus type 1 thymidine kinase (TK) protein including a first TK self complementing fragment and a first target protein, wherein the first TK self complementing fragment comprises a first portion of a TK protein; expressing the first polynucleotide to produce the first TK protein; providing a second vector that includes a second polynucleotide sequence that encodes a second TK protein including a second TK self complementing fragment and a second target protein, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein; expressing the second polynucleotide to produce the second TK protein; providing a labeled TK substrate, wherein the label of the labeled TK substrate being able to generate a signal; and generating a signal from the label if the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

Another exemplary method of detecting protein-protein interaction, among others, includes: providing a first herpes simplex virus type 1 thymidine kinase (TK) protein, wherein the first TK protein includes a first TK self complementing fragment and a first target protein, wherein the first TK self complementing fragment comprises a first portion of a TK protein; providing a second TK protein, wherein the second TK protein includes a second TK self complementing fragment and a second target protein, wherein the second TK self complementing fragment comprises a second portion of the TK protein that is complementary with the first self complementing fragment, wherein the first TK self complementing fragment and the second TK self complementing fragment are not active individually, and wherein the first TK self complementing fragment and the second TK self complementing fragment spontaneously self complement to substantially form an active TK protein; providing a labeled TK substrate, wherein the label of the labeled TK substrate being able to generate a signal; and generating a signal from the label if the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

Another exemplary method of detecting protein-protein interaction, among others, includes: providing a first herpes simplex virus type 1 thymidine kinase (TK) protein, wherein the first TK protein includes a first TK self complementing fragment and a first target protein, wherein the first TK self complementing fragment comprises a first portion of a TK protein; exposing the first TK protein to a cell, wherein the cell comprises a second TK protein, wherein the second TK protein includes a second TK self complementing fragment and a second target protein, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein; introducing a labeled TK substrate to the cell, wherein the label of the labeled TK substrate being able to generate a signal; and generating a signal from the label if the first target protein enter the cell and the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

One exemplary method of cellular localization of proteins, among others, includes: providing a first herpes simplex virus type 1 thymidine kinase (TK) protein, wherein the first TK protein includes a first TK self complementing fragment and a first target protein, wherein the first TK self complementing fragment comprises a first portion of a TK protein; exposing the first protein to a cell, wherein a compartment of the cell comprises a second TK protein, wherein the second TK protein includes a second TK self complementing fragment and a second target protein, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein; introducing a labeled TK substrate to the cell, wherein the label of the labeled TK substrate being able to generate a signal; and generating a signal from the label if the first target protein enters the compartment of the cell and the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

One exemplary fusion protein, among others, includes: a TK protein including a TK self complementing fragment and a target, wherein the TK self complementing fragment comprises a first portion of a TK protein.

These embodiments, uses of these embodiments, and other uses, features and advantages of the present disclosure, will become more apparent to those of ordinary skill in the relevant art when the following detailed description of the preferred embodiments is read in conjunction with the appended figures.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1a is a ribbon diagram of the quaternary structure of the HSV1-TK homodimer. The constituent subunits of TK (376 amino acids long) display the general aβ folding pattern. Each aβ structure is made up of 15 α-helices and 7 β-sheets. A 5-stranded parallel β-sheet forms part of the core of the molecule, which contains 5 active sites.

FIG. 1b is a schematic diagram showing the protein-fragment-assisted complementation strategy using split HSV1-TK (here abbreviated as TK) to monitor the hypothetical X-Y heterodimeric protein-protein interaction. This is accomplished by fusing each of the reporter fragments to heterologous X-Y protein domains to generate two chimeric proteins that have the capacity to interact with one another. If the interaction of the two heterologous protein domains (first and foremost) restores the activity of the reporter by bringing the two reporter fragments into close spatial proximity (as a secondary consequence), then this restoration of reporter activity can be used to monitor the interaction of the two heterologous protein domains. Dimerization of the two proteins restores TK activity through protein complementation and produces a PET imaging signal in the presence of radiolabeled TK substrate. If the reporter protein is an enzyme, then an additional strength of this PCA approach is the capacity to amplify the signal associated with each protein-protein interaction event.

FIG. 2a illustrates a schematic representation of the plasmid vector constructs made for transient expression of the seven genes transfected individually or in combinations described in the text and Supplementary Methods section, for evaluation of the PCA strategy. Each vector was cloned into a pcDNA3.1 (+) plasmid backbone, under control of a CMV promoter.

FIG. 2b illustrates a schematic diagram of the intramolecular folding sensor construct with the split TK fragments on either side of the estrogen receptor ligand binding domain (ER-LBD), and each construct was made by cloning into a pcDNA3.1 (+) plasmid backbone, under control of a CMV promoter. 293T cells were transiently transfected (500 ng DNA per well) to express the intramolecular folding sensor and treated with the indicated ER ligands or carrier control (dymethyl sulfoxide, DMSO) for 24 h, followed by measurement of TK enzyme uptake (expressed as normalized dpm of cells/dpm in medium/microgram of protein). Treatment with the ER ligands 17β-estradiol (E2), 4-hydroxytamoxifen (4-OHT), raloxifene, methyl piperidinylethoxy pyrazole (MPP), diethylstilbestrol (DES), and 1,3,5-tris(4-hydroxyphenyl)-4-propyl-1H-pyrazole (PPT) led to levels of intramolecular-folding-assisted complementation that were significantly higher than that of carrier control-treated cells (P<0.05) except for genistein. The error bar is the standard error of the mean for three samples. Western blot analysis using anti-ERα antibody after treatment with the different ligands shows adequate expression levels in 293T cells.

FIG. 2c is a schematic representation of the plasmid vector constructs made for transient expression of the eight genes transfected in combinations described in the text, for evaluation of the relative orientation of reporter fragments and interacting proteins on the functioning of the PCA strategy. Each construct was made by cloning into a pcDNA3.1 (+) plasmid backbone, under control of a CMV promoter.

FIG. 2d is a graph to show comparison of coexpressed chimeras carrying nTK or cTK with FRB/FKBP12 (with and without rapamycin), to show the effect of relative orientation of reporter fragments and interacting proteins on enzyme activity of a PCA, measured by TK enzyme uptake (expressed as normalized dpm of cells/dpm in medium/microgram of protein) in transiently transfected 293T cells, with mock (negative) and full length HSV1-sr39TK (positive) controls. The error bar is the standard error of the mean for three samples. Letters denoting the various chimeras are as shown in panel FIG. 2c. There was a statistically significant increase in measured TK activity after co-transfecting chimeras F with G, upon addition of rapamycin.

FIGS. 3a-3d provide an evaluation of plasmid vector constructs containing the V119C mutation. FIG. 3a is a schematic representation of the plasmid vector constructs made for transient expression of the four genes transfected in combinations described in the text, for evaluation of the PCA strategy after introducing point mutations V119C and R318c in the TK gene. Each vector was cloned into a pcDNA3.1 (+) plasmid backbone, under control of a CMV promoter.

FIG. 3b is a graph to show comparison of coexpressed chimeras carrying nTK or cTK with FRB/FKBP12 (with and without rapamycin), and chimeras containing the TK point mutations V119C and R318c on enzyme activity in a PCA, measured by TK enzyme uptake (expressed as normalized dpm of cells/dpm in medium/microgram of protein) in transiently transfected 293T cells, with mock (negative) and full length HSV1-sr39TK (positive) controls. The error bar is the standard error of the mean for three samples. Introducing the point mutation V119C to the nTK fragment resulted in an increase (at limit of statistical significance) in measured TK activity upon addition of rapamycin, after cotransfecting nTK_{(V119C)}-FRB and FKBP12-cTK (Vectors II and III). NS: not significant.

FIG. 3c is a schematic representation of the single plasmid vector construct made for stable expression of the four genes (two split TK and two interacting protein fragments) driven by pUbi and pCMV promoters, for evaluation of the PCA strategy after introducing the point mutation V119C in the nTK gene. This vector was cloned into a pcDNA3.1 (+) plasmid backbone.

FIG. 3d illustrates the expression levels of FRB (mTOR) and FKBP12 (in their endogenous and fusion forms) were determined in total lysates of cells transfected with the single vector in panel FIG. 3c by immunoblotting with the corresponding antibodies (anti-TK, -FRB, -FKBP12). Anti-α-tubulin was used as an internal control for loading. Cells were exposed to 40 nM rapamycin. Anti-mTOR did not reveal a band for expression of nTK-FRB (despite strong α-tubulin expression) likely because of the much smaller size of FRB as compared with the entire mTOR protein.

FIG. 4a is a graph to show in vitro response of 293T cells stably transfected with pcDNA-pUbi-FKBP12-cTK-pCMV-nTK_{(V119C)}-FRB after 36 h exposure to escalating doses of rapamycin, as measured by TK enzyme uptake (expressed as normalized dpm of cells/dpm in medium/microgram of protein).

FIG. 4b is a graph to show in vitro response of 293T cells stably transfected with pcDNA-pUbi-FKBP12-cTK-pCMV-nTK_{(V119C)}-FRB after 36 h exposure to escalating doses of ascomycin (FK506) along with a fixed 40 nM of rapamycin, as measured by TK enzyme uptake (expressed as normalized dpm of cells/dpm in medium/microgram of protein). An increasing concentration of ascomycin led to a reduction in TK complementation, likely because it blocked the binding of rapamycin to FKBP12 and thus reduced the heterodimerization with FRB.

FIGS. 5a-5c illustrate imaging of tumors containing the split TK constructs. FIG. 5a is a transaxial tomographic microPET images through a representative prone-positioned mouse implanted subcutaneously over the left shoulder with mock transfected 293T cells, and over the right shoulder with 293T cells stably expressing both nTK_{(V119C)}-FRB and FKBP12-cTK. The mouse was injected with 200 μCi of [¹⁸F]-FHBG prior to imaging on days 1, 2, and 5 into the imaging protocol (i.e., after 7 days of initial xenograft growth). Elliptical dotted white line outlines the surface of the mouse's upper thorax. Color intensity is a reflection on probe accumulation after its phosphorylation by the complemented TK enzyme. Quantitative analysis of this probe accumulation shows a mean % ID/g (obtained from 5 tomographic slices through each tumor for all animals) as displayed in accompanying graph FIG. 5b. The difference between accumulation in tumors exhibiting split TK complementation and control tumors was statistically significant (P=0.02) on Day 5. Also shown is the optical CCD imaging on day 5 of the imaging protocol of the same mouse immediately before its subsequent microPET imaging of bilateral shoulder region subcutaneous xenografts, and is shown as a visible light image superimposed on the CCD bioluminescence image with a scale in photons/sec/cm²/steradian. Mice were imaged in the prone position after tail-vein injection of 4 mg D-Luciferin per animal. Each mouse was implanted subcutaneously over the left shoulder with 5×10⁶ mock transfected 293T cells admixed with 50,000 293T stable cells expressing Firefly luciferase, and subcutaneously over the right shoulder with 5×10⁶ 293T stable cells expressing both nTK_{(V119C)}-FRB and FKBP12-cTK admixed with 50,000 293T stable cells expressing Firefly luciferase. Bioluminescence imaging shows equivalent viable tumor load in both xenografts. Angled black dotted line shows the transaxial plane, bisecting each tumor, through which the microPET images were obtained. FIG. 5c is a coronal tomographic microPET images through two representative prone mice implanted subcutaneously over the left shoulder (circle 1) with control tumors of 293T cells stably expressing nTK plus cTK only (in a single vector), and over the right shoulder (circle 2) with 293T cells expressing nTK_{(V119C)}-FRB plus FKBP12-cTK in a single vector. Unlike tumors containing the complemented TK enzyme, there was minimal FHBG accumulation (% ID/g) in the control tumors on the fifth day of the imaging protocol upon systemic administration of rapamycin (see text). Intense accumulation in centre of image is due to non-specific probe excretion in the gut.

FIG. 6 illustrates the structure and circular permutation strategy for TK. Ribbon diagrams of five TK molecules rotated in space to show positions of the five chosen split sites (yellow arrows) in this Example. These split sites are numbered in red and are also bracketed (in red) onto the primary sequence of TK. This is to demonstrate the relative positions of the split sites to β-pleated sheets (blue), a-helices (green), and the active sites of the enzyme (underlined). TK is 376 amino acids long.

FIG. 7 illustrates the circular permutation strategy for TK. FIG. 7 illustrates the scheme of the construction of the five circularly permuted genes of HSV1-TK. cDNA fashioned from PCR products cTK and nTK are joined by a linker region. Each vector was cloned into a pcDNA3.1 (+) plasmid backbone, under control of a CMV promoter. The original protein termini are covalently linked to form a circular polypeptide. The new termini are then created by cleavage of a peptide bond at a location along the backbone distant from the original termini.

FIG. 8 illustrates a graph to show enzyme activity of five circularly permuted variants of TK, measured by TK enzyme uptake (expressed as normalized dpm of cells/dpm in medium/microgram of protein) in transiently transfected 293T cells, with mock (negative) and full length HSV1-sr39TK (positive) controls. The error bar is the standard error of the mean for three samples.

FIG. 9 illustrates a graph to show time course of enzyme activity of a PCA using coexpressed chimeras carrying nTK or cTK with FRB/FKBP12 (with and without rapamycin), measured by TK enzyme uptake (expressed as normalized dpm of cells/dpm in medium/microgram of protein) in transiently transfected 293T cells, with mock (negative) and full length HSV1-sr39TK (positive) controls. The error bar is the standard error of the mean for three samples.

FIG. 10 illustrates a graph to show comparison of coexpressed chimeras carrying nTK or cTK with FRB/FKBP12 (with and without rapamycin), to study the effect of rapamycin dosage on enzyme activity of a PCA, measured by TK enzyme uptake (expressed as normalized dpm of cells/dpm in medium/microgram of protein) in transiently transfected 293T cells, with mock (negative) and full length HSV1-sr39TK (positive) controls. The error bar is the standard error of the mean for three samples.

FIG. 11 illustrates a graph to show comparison of coexpressed chimeras carrying nTK or cTK with FRB/FKBP12 (with and without rapamycin) in different cell lines, as measured by TK enzyme uptake (expressed as normalized dpm of cells/dpm in medium/microgram of protein) in transiently transfected 293T cells, SKBr3 cells, and SKOV3 cells. The error bar is the standard error of the mean for three samples. There was a statistically significant increase in measured TK activity upon addition of rapamycin to all 3 cell lines. Western blot analysis of all 3 cells using anti-TK antibody before and after addition of rapamycin.

FIG. 12 illustrates a graph to show comparison of coexpressed chimeras carrying nTK or cTK with FRB/FKBP12 (with and without rapamycin), Id/MyoD, and nTK expressed with cTK (without interacting proteins) on enzyme activity of a PCA, measured by TK enzyme uptake in transiently transfected 293T cells, with mock, nTK alone, and cTK alone as negative controls, and full length HSV1-sr39TK as positive controls. The error bar is the standard error of the mean for three samples.

FIG. 13 illustrates a graph to show comparison of coexpressed chimeras carrying nTK or cTK with FRB/FKBP12 (with and without rapamycin), Id/MyoD, and deliberately mismatched two other sets of chimeras on enzyme activity of a PCA, measured by TK enzyme uptake in transiently transfected 293T cells, with mock (negative) and full length HSV1-sr39TK (positive) controls. The error bar is the standard error of the mean for three samples.

FIG. 14 illustrates a graph to show reversibility of the split TK-based PCA using a ligand-reversible dimer of a mutant FKBP12 called F_{M} (F36M) that can be disrupted by FK506. We fused F_{M} to split TK fragments and transiently expressed the resulting chimeras, nTK_{(V119C)}-F_{M} and F_{M}-cTK, in a single vector in 293T cells. Twenty-four hours later we added FK506 to these cells and, using an in vitro [8-³H]Penciclovir cell uptake assay, measured the time-dependent changes in homodimer complex dissociation and consequent diminished TK complementation. Five μM FK506 resulted in dissociation of about two-thirds of complexes within 30 min. The extent of TK complementation using the F_{M} homodimer (with no added FK506) was compared initially with that achieved following FRB/FKBP12/rapamycin (Rap) heterodimerization. The control 293T cells carried an empty vector. The error bar is the standard error of the mean for three samples.

FIG. 15 illustrates a graph to show reversibility of the split TK-based PCA using a ligand-reversible dimer of a mutant FKBP12 called F_{M} (F36M) that can be disrupted by FK506. We fused F_{M} to split TK fragments and transiently expressed the resulting chimeras, nTK_{(V119C)}-F_{M} and F_{M}-cTK, in a single vector in 293T cells. Twenty-four hours later we added FK506 to these cells and, using an in vitro [8-³H]Penciclovir cell uptake assay, measured the dose-dependent changes in homodimer complex dissociation and consequent diminished TK complementation. Incremental reduction of TK activity was observed with increasing doses of FK506 up to 5 μM (measured after 24 hr of exposure to this ligand). Control 293T cells carried an empty vector. The error bar is the standard error of the mean for three samples.

FIG. 16 illustrates fluorescence micrographs (×400 magnification) after immunohistochemical staining of control and experimental tumors shows considerable levels of TK protein expression from the tumors developed from 293T cells stably expressing split TK plus the interacting proteins, and not from the control mock transfected 293T cells.

FIG. 17 illustrates the relative advantage of PET over optical imaging in imaging sources of signal at depths below 1 cm from the exterior is exemplified in this separate experiment, where mice were imaged 6 days post implantation with 5×10⁶ 293T cells stably expressing TK or Firefly luciferase (FLUC). The microPET signal return (FHBG accumulation in % ID/g) from shoulder tumors (circled) expressing TK did not differ whether the animals were imaged in the supine or prone positions, as demonstrated in the transaxial and coronal tomographic images (intense accumulation in centre and lower aspect of coronal images is due to non-specific probe excretion in the gut). When imaged in the supine position, the light emanating from subcutaneous xenografts on the shoulder/back showed no penetration through the full thickness of the mouse.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “complementing fragments” or “complementary fragments” when used in reference to a reporter polypeptide refer to fragments of a polypeptide that are individually inactive (e.g., do not express the reporter phenotype), wherein binding of the complementing fragments restores reporter activity. The terms “self-complementing”, “self-assembling”, and “spontaneously-associating”, when used to describe two fragments of the same protein, mean that the fragments are capable of reconstituting into an active protein when the individual fragments are soluble and are sufficiently close to or contact one another.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions that determine whether a nucleic acid is specifically hybridized to a surface bound nucleic acid. Wash conditions used to identify nucleic acids may include (e.g., a salt concentration of about 0.02 molar at pH 7 and a temperature of at least about 50° C. or about 55° C. to about 60° C.; or, a salt concentration of about 0.15 M NaCl at 72° C. for about 15 mins; or, a salt concentration of about 0.2×SSC at a temperature of at least about 50° C. or about 55° C. to about 60° C. for about 15 to about 20 mins; or, the hybridization complex is washed twice with a solution with a salt concentration of about 2×SSC containing 0.1% SDS at room temperature for 15 mins and then washed twice by 0.1×SSC containing 0.1% SDS at 68° C. for 15 mins; or, equivalent conditions). Stringent conditions for washing can also be (e.g., 0.2×SSC/0.1% SDS at 42° C.).

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “salts” herein refers to both salts of carboxyl groups and to acid addition salts of amino groups of the polypeptides of the present disclosure. Salts of a carboxyl group may be formed by methods known in the art and include inorganic salts, for example, sodium, calcium, ammonium, ferric or zinc salts, and the like, and for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

Several methods are known in the art for incorporating non-naturally occurring amino acid residues into proteins, and a few are described below.

For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography.

In a second method, translation is carried out in Xenopus oocytes by microinjection of mutated mRNA and chemically aminoacylated suppressor tRNAs (Turcatti, et al., J. Biol. Chem., 271: 19991-8, 1996).

In a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

A “fragment” of a molecule such as a protein or nucleic acid is meant to refer to any portion of the amino acid or nucleotide genetic sequence.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this disclosure, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. Polynucleotide encompasses the terms “nucleic acid,” “nucleic acid sequence,” or “oligonucleotide” as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process.

By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alias.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” means a specific triplet of mononucleotides in the DNA chain. Codons correspond to specific amino acids (as defined by the transfer RNAs) or to start and stop of translation by the ribosome.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

The DNA encoding the protein disclosed herein can be prepared by the usual methods: cloning cDNA from mRNA encoding the protein, isolating genomic DNA and splicing it, chemical synthesis, and so on.

cDNA can be cloned from mRNA encoding the protein by, for example, the method described below:

First, the mRNA encoding the protein is prepared from the above-mentioned tissues or cells expressing and producing the protein. mRNA can be prepared by isolating total RNA by a known method such as guanidine-thiocyanate method (Chirgwin et al., Biochemistry, 18:5294, 1979), hot phenol method, or AGPC method, and subjecting it to affinity chromatography using oligo-dT cellulose or poly-U Sepharose.

Then, with the mRNA obtained as a template, cDNA is synthesized, for example, by a well-known method using reverse transcriptase, such as the method of Okayama et al (Mol. Cell. Biol. 2:161 (1982); Mol. Cell. Biol. 3:280 (1983)) or the method of Hoffman et al. (Gene 25:263 (1983)), and converted into double-stranded cDNA. A cDNA library is prepared by transforming E. coli with plasmid vectors, phage vectors, or cosmid vectors having this cDNA or by transfecting E. coli after in vitro packaging.

The plasmid vectors used herein are not limited as long as they are replicated and maintained in hosts. Any phage vector that can be replicated in hosts can also be used. Examples of usually used cloning vectors are pUC19, gt10, gt11, and so on. When the vector is applied to immunological screening as mentioned below, a vector having a promoter that can express a gene encoding the desired protein in a host is preferably used.

cDNA can be inserted into a plasmid by, for example, the method of Maniatis et al. (Molecular Cloning, A Laboratory Manual, second edition, Cold Spring Harbor Laboratory, p. 1.53, 1989). cDNA can be inserted into a phage vector by, for example, the method of Hyunh et al. (DNA cloning, a practical approach, 1, p. 49 (1985)). These methods can be simply performed by using a commercially available cloning kit (e.g., a product from Takara Shuzo). The recombinant plasmid or phage vector thus obtained is introduced into an appropriate host cell such as a prokaryote (e.g., E. coli: HB101, DH5a, MC1061/P3, etc).

Examples of a method for introducing a plasmid into a host (e.g., cell) are, calcium chloride method, calcium chloride/rubidium chloride method and electroporation method, described in Molecular Cloning, A Laboratory Manual (second edition, Cold Spring Harbor Laboratory, p. 1.74 (1989)). Phage vectors can be introduced into host cells by, for example, a method in which the phage DNAs are introduced into grown hosts after in vitro packaging. In vitro packaging can be easily performed with a commercially available in vitro packaging kit (for example, a product from Stratagene or Amersham). Genes can also be introduced into a host using viral and non-viral vectors.

The identification of cDNA encoding protein, its expression being augmented depending on the stimulation of cytokines like AID protein disclosed herein, can be carried out by for example suppression subtract hybridization (SSH)(Proc. Natl. Acad. Sci. USA, 93:6025-6030, 1996; Anal. Biochem., 240:90-97, 1996) taking advantage of suppressive PCR effect (Nucleic Acids Res., 23:1087-1088, 1995), using two cDNA libraries, namely, cDNA library constructed from mRNA derived from stimulated cells (tester cDNA library) and that constructed from mRNA derived from unstimulated cells (driver cDNA library).

Specific examples of the vectors for recombination used are E. coli-derived plasmids such as pBR322, pBR325, pUC12, pUC13, and pUC19, yeast-derived plasmids such as pSH19 and pSH15, and Bacillus subtilis-derived plasmids such as pUB110, pTP5, and pC194. Examples of phages are a bacteriophage such as phage, and an animal or insect virus (pVL1393, Invitrogen) such as, a retrovirus, a vaccinia virus, and a nuclear polyhedrosis virus.

An “expression vector” is useful for expressing the DNA encoding the protein used herein and for producing the protein. The expression vector is not limited as long as it expresses the gene encoding the protein in various prokaryotic and/or eukaryotic host cells and produces this protein. Examples thereof are pMAL C2, pEF-BOS (Nucleic Acids Res. 18:5322 (1990) and so on), pME18S (Experimental Medicine: SUPPLEMENT, “Handbook of Genetic Engineering” (1992)), etc.

When bacteria, particularly E. coli are used as host cells, an expression vector generally comprises, at least, a promoter/operator region, an initiation codon, the DNA encoding the protein termination codon, terminator region, and replicon.

When yeast, animal cells, or insect cells are used as hosts, an expression vector is preferably comprising, at least, a promoter, an initiation codon, the DNA encoding the protein and a termination codon. It may also comprise the DNA encoding a signal peptide, enhancer sequence, 5′- and 3′-untranslated region of the gene encoding the protein, splicing junctions, polyadenylation site, selectable marker region, and replicon. The expression vector may also contain, if required, a gene for gene amplification (marker) that is usually used.

A promoter/operator region to express the protein in bacteria comprises a promoter, an operator, and a Shine-Dalgarno (SD) sequence (e.g., AAGG). For example, when the host is Escherichia, it preferably comprises Trp promoter, lac promoter, recA promoter, lambda PL promoter, b 1 pp promoter, tac promoter, or the like. Examples of a promoter to express the protein in yeast are PH05 promoter, PGK promoter, GAP promoter, ADH promoter, and so on. When the host is Bacillus, examples thereof are SL01 promoter, SP02 promoter, penP promoter, and so on. When the host is a eukaryotic cell such as a mammalian cell, examples thereof are SV40-derived promoter, retrovirus promoter, heat shock promoter, and so on, and preferably SV-40 and retrovirus-derived one. As a matter of course, the promoter is not limited to the above examples. In addition, using an enhancer is effective for expression.

A preferable initiation codon is, for example, a methionine codon (ATG).

A commonly used termination codon (e.g., TAG, TAA, TGA) is exemplified as a termination codon. Usually, used natural or synthetic terminators are used as a terminator region.

A “replicon” means a DNA capable of replicating the whole DNA sequence in host cells, and includes a natural plasmid, an artificially modified plasmid (DNA fragment prepared from a natural plasmid), a synthetic plasmid, and so on. Examples of preferable plasmids are pBR322 or its artificial derivatives (DNA fragment obtained by treating pBR322 with appropriate restriction enzymes) for E. coli, yeast plasmid or yeast chromosomal DNA for yeast, and pRSVneo ATCC 37198, pSV2dhfr ATCC 37145, pdBPV-MMTneo ATCC 37224, pSV2neo ATCC 37149, and such for mammalian cells.

An enhancer sequence, polyadenylation site, and splicing junction that are usually used in the art, such as those derived from SV40 can also be used.

A selectable marker usually employed can be used according to the usual method. Examples thereof are resistance genes for antibiotics, such as tetracycline, ampicillin, or kanamycin.

Examples of genes for gene amplification are dihydrofolate reductase (DHFR) gene, thymidine kinase gene, neomycin resistance gene, glutamate synthase gene, adenosine deaminase gene, ornithine decarboxylase gene, hygromycin-B-phophotransferase gene, aspartate transcarbamylase gene, etc. It should also be noted that these are also selection genes, except for use in mammalian cells instead of the genes described in the paragraph above, which are used in bacteria. Usually the genes described in the paragraph above are used for plasmid amplification in bacterial cells and the ones in this paragraph are used for selection of mammalian cells.

The expression vector used herein can be prepared by continuously and circularly linking at least the above-mentioned promoter, initiation codon, DNA encoding the protein, termination codon, and terminator region, to an appropriate replicon. If desired, appropriate DNA fragments (for example, linkers, restriction sites, and so on), can be used by the usual method such as digestion with a restriction enzyme or ligation using T4 DNA ligase.

As used herein, “transformants” can be prepared by introducing the expression vector mentioned above into host cells.

As used herein, “host” cells are not limited as long as they are compatible with an expression vector mentioned above and can be transformed. Examples thereof are various cells such as wild-type cells or artificially established recombinant cells usually used in technical field (e.g., bacteria (Escherichia and Bacillus), yeast (e.g., Saccharomyces, Pichia, and such), animal cells, or insect cells).

Specific examples are E. coli (e.g., DH5ₐₗₚₕₐ, TB1, HB101, and such), mouse-derived cells (e.g., COP, L, C127, Sp2/0, NS-1, NIH 3T3, and such), rat-derived cells (e.g., PC12, PC12h), hamster-derived cells (e.g., BHK, CHO, and such), monkey-derived cells (e.g., COS1, COS3, COS7, CV1, Velo, and such), and human-derived cells (Hela, diploid fibroblast-derived cells, myeloma cells, and HepG2, and such).

An expression vector can be introduced (transformed/transfected/transduced/electroporated) into host cells by known methods.

Transformation can be performed, for example, according to the method of Cohen et al. (Proc. Natl. Acad. Sci. USA, 69:2110 (1972)), protoplast method (Mol, Gen. Genet., 168:111 (1979)), or competent method (J. Mol. Biol., 56:209 (1971)) when the hosts are bacteria (E. coli, Bacillus subtilis, and such), the method of Hinnen et al. (Proc. Natl. Acad. Sci. USA, 75:1927 (1978)), or lithium method (J. Bacteriol., 153:163 (1983)) when the host is Saccharomyces cerevisiae, the method of Graham (Virology, 52:456 (1973)) when the hosts are animal cells, and the method of Summers et al. (Mol. Cell. Biol., 3:2156-2165 (1983)) when the hosts are insect cells.

The protein disclosed herein, can be produced by cultivating transformants (in the following, this term includes transfectants) comprising an expression vector prepared as mentioned in nutrient media.

The nutrient media preferably comprise carbon source, inorganic nitrogen source, or organic nitrogen source necessary for the growth of host cells (transformants). Examples of the carbon source are glucose, dextran, soluble starch, and sucrose, and examples of the inorganic or organic nitrogen source are ammonium salts, nitrates, amino acids, corn steep liquor, peptone, casein, meet extract, soy bean cake, and potato extract. If desired, they may comprise other nutrients (for example, an inorganic salt (for example, calcium chloride, sodium dihydrogenphosphate, and magnesium chloride), vitamins, antibiotics (for example, tetracycline, neomycin, ampicillin, kanamycin, and so on).

Cultivation of cell lines is performed by a method known in the art. Cultivation conditions such as temperature, pH of the media, and cultivation time are selected appropriately so that the protein is produced in large quantities.

Examples of the isolation and purification method are a method utilizing solubility, such as salting out and solvent precipitation method; a method utilizing the difference in molecular weight, such as dialysis, ultrafiltration, gel filtration, and sodium dodecyl sulfate-polyacrylamide gel electrophoresis; a method utilizing charges, such as ion exchange chromatography and hydroxylapatite chromatography; a method utilizing specific affinity, such as affinity column chromatography; a method utilizing the difference in hydrophobicity, such as reverse phase high performance liquid chromatography; and a method utilizing the difference in isoelectric point, such as isoelectric focusing.

As used herein, the term “hybridization” refers to the process of association of two nucleic acid strands to form an antiparallel duplex stabilized by means of hydrogen bonding between residues of the opposite nucleic acid strands.

The term “immunologically active” defines the capability of the natural, recombinant or synthetic bioluminescent protein, or any oligopeptide thereof, to induce a specific immune response in appropriate animals or cells and to bind with specific antibodies. As used herein, “antigenic amino acid sequence” means an amino acid sequence that, either alone or in association with a carrier molecule, can elicit an antibody response in a mammal. The term “specific binding,” in the context of antibody binding to an antigen, is a term well understood in the art and refers to binding of an antibody to the antigen to which the antibody was raised, but not other, unrelated antigens.

As used herein the term “isolated” is meant to describe a polynucleotide, a polypeptide, an antibody, or a host cell that is in an environment different from that in which the polynucleotide, the polypeptide, the antibody, or the host cell naturally occurs.

“Optional” or “optionally” means that the subsequently described circumstance may or may not occur, so that the description includes instances where the circumstance occurs and instances where it does not.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids (e.g., surface bound and solution phase nucleic acids) of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the disclosure can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the present disclosure or their analogs.

As used herein, the term “imaging probe”, “imaging agent”, or “imaging compound” refers to the labeled compounds of the present disclosure that are capable of serving as imaging agents and whose uptake is related to the expression level of certain surface cell receptors (e.g., integrin αᵥβ₃). In particular non-limiting embodiments the imaging probes or imaging agents of the present disclosure are labeled with a PET isotope, such as F-18.

By “administration” is meant introducing a compound of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “a detectably effective amount” of the imaging agent of the present disclosure is defined as an amount sufficient to yield an acceptable image using equipment that is available for clinical use. A detectably effective amount of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The term “therapeutically effective amount” as used herein refers to that amount of the compound being administered which will relieve to some extent one or more of the symptoms of a disease, a condition, or a disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

“Treating” or “treatment” of a disease (or a condition or a disorder) includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms also mean that the life expectancy of an individual affected with a cancer will be increased or that one or more of the symptoms of the disease will be reduced.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to host or organisms noted above that are alive and are not dead. The term “living host” refers to the entire host or organism and not just a part excised (e.g., a liver or other organ) from the living host.

The term “sample” can refer to a tissue sample, cell sample, a fluid sample, and the like. The sample may be taken from a host. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “detectable signal” is a signal derived from a labeled TK substrate. The detectable signal is detectable and distinguishable from other background signals that may be generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the acoustic detectable signal and/or the background.

### General Discussion

In general, the present disclosure includes split protein herpes simplex virus type 1 thymidine kinase [HSV1-TK or TK] TK fragments, split protein TK systems, methods of imaging protein-protein interactions, methods of cellular localization of proteins, methods of evaluating protein translocation and trafficking, and the like. In addition, the present disclosure includes compositions used in and methods relating to non-invasive imaging (e.g., positron emission tomography (PET) imaging), in vivo and/or in vitro. Additional details regarding the imaging compound are described in Example 1.

In general, a split protein TK system includes a first TK self complementing fragment (which may be part of a first TK protein) and a second TK self complementing fragment (which may be part of a second TK protein) that are initially separated. The first TK self complementing fragment and the second TK self complementing fragment can be provided to a host or system. The term “provided” can mean that the fragments are administered, delivered, injected, or the like, to the host or system and/or that the fragments can be expressed in the host or system using an appropriate expression system (e.g., a vector), as is appropriate for the context that the term is used.

If the first TK self complementing fragment and the second TK self complementing fragment come into close proximity or into contact with one another, they spontaneously self complement (e.g., inherent self affinity between the fragments protein brings the fragments close to each other and generates an event called complementation) to form an active TK protein that acts the same as or similar to the intact TK protein that has not been split. The first TK self complementing fragment and the second TK self complementing fragment are not active like the intact TK protein. The term “active” in active TK protein refers to the ability of the active TK protein to interact with a TK substrate (e.g., a labeled TK substrate) to modify (e.g., phosphorylation) the TK substrate so that the modified TK substrate can be detected.

In an embodiment, the first TK self complementing fragment and the second TK self complementing fragment can be disposed near one another through the protein-protein interaction of a first protein (associated with the first TK self complementing fragment) and a second protein (associated with the second TK self complementing fragment). The active TK protein formed from the complementation of the first TK self complementing fragment and the second TK self complementing fragment can interact with a labeled TK substrate. The labeled TK substrate can be chemically modified by an interaction (e.g., phosphorylation) with the active TK protein so that the altered labeled TK substrate is trapped in the cell. Thus, the altered labeled TK substrate can accumulate in the cell(s) and be detected (e.g., the label is a PET label and the detection system is a PET system). In an embodiment, the detection of the altered labeled TK substrate indicates interaction of the first protein and the second protein noted above.

The term “complementing fragment” or “complementary fragment” when used in reference to the TK fragments (first and second) refer to fragments of polypeptides that are individually inactive (e.g., do not express the reporter phenotype), where binding of the complementing fragments partly or completely restores reporter activity. The terms “self-complementing”, “self-assembling”, and “spontaneously-associating”, when used to describe two fragments of the same protein, mean that the fragments are capable of reconstituting into an active TK protein when the individual fragments are soluble and are sufficiently close to or contact one another.

In an embodiment, a first TK self complementing fragment (N-terminal fragment of the TK, SEQ ID No: 1, amino acids 1-265) is associated (e.g., biologically (e.g., bound to or expressed in), chemically (e.g., ionic bond, covalent bond, hydrogen bonding, and the like), and/or physically) to a first target or system (e.g., a protein, a peptide, a cell (e.g., inside of or outside of), an organelle, a drug, a macromolecule, and the like), while a second TK self complementing fragment (C-terminal fragment of the TK, SEQ ID No: 1, amino acids 266-376) is associated with a second target or system (e.g., a protein, a peptide, a cell (e.g., inside of or outside of), an organelle, a drug, a macromolecule, and the like). The first TK self complementing fragment and the second TK self complementing fragment are not active like the intact TK protein in that they do not interact with the TK substrate to modify the TK substrate as described herein. The first and second TK self complementing fragments can be introduced into a system (e.g. inside a cell or outside a cell) and/or the first and/or the TK second self complementing fragments can be expressed (e.g., using a vector or other expression system including SEQ ID No: 2, nucleotides 1-795, and nucleotides 796-1125 that correspond to the first and the TK second self complementing fragments, respectively) in the system.

Then, if the first TK self complementing fragment and the second TK self complementing fragment come into contact with one another as a result of the first target and second target interacting with one another, the TK fragments spontaneously self complement (e.g., inherent self affinity between the N- and C-terminal fragments of a monomeric TK protein brings the fragments close to each other and generates an event called complementation) to form the active TK protein. As noted above, the active TK protein can interact with a labeled TK substrate. A signal from the modified labeled TK substrate can be subsequently detected. In an embodiment, the signal may need to be a sum of each of the individual modified labeled TK substrates. In an embodiment, the signal can be generated from a summation, an integration, or other mathematical process, formula, or algorithm, where the signal is from one or more modified labeled TK substrates. In an embodiment, the summation, the integration, or other mathematical process, formula, or algorithm can be used to generate the signal so that the signal can be distinguished from background noise and the like.

In an embodiment, the split protein TK self complementing fragments are used in methods of detecting protein-protein interaction. The first TK protein includes, but is not limited to, a first target protein and a first TK self complementing fragment. The first TK self complementing fragment includes a first portion of a TK protein (SEQ ID No: 1), as described in more detail herein. The second TK protein includes, but is not limited to, a second target protein and a second TK self complementing fragment. The second TK self complementing fragment includes a second portion of a TK protein (SEQ ID No: 1). The first target protein and the second target protein can be proteins of interest in regard to a protein-protein interaction in a system. If the first protein and the second protein interact with one another, the first TK self complementing fragment and the second TK self complementing fragment can spontaneously self complement to form an active TK protein. The active TK protein can interact with a labeled TK substrate. The labeled TK substrate can be modified (e.g., phosphorylated) and can accumulate in the cell, for example. The modified labeled TK substrate(s) can produce a signal and subsequently be detected. Therefore, protein-protein interactions can be detected, studied, monitored, and/or evaluated.

In another embodiment, the split protein TK self complementing fragments are used in methods of the cellular localization of proteins. This system provides an easy and a quantitative readout when compared to other techniques in use for this purpose. Even though much study has been conducted to understand the functional aspects of cells, still many questions remain. In addition, many cellular networks function by movement of proteins from one compartment to another. Embodiments of the split protein TK self complementing fragments and systems can be used to identify nucleocytoplasmic and intra- and inter-compartment movement of proteins inside the cell.

In general, the method includes, but is not limited to, a first TK protein and a second TK protein. The first TK protein includes, but is not limited to, a first target protein and a first TK self complementing fragment. The first TK self complementing fragment includes a first portion (e.g., C-terminal fragment of SEQ ID No: 1) of a TK protein, as described in more detail herein. The first TK protein is exposed to a cell. A compartment (e.g., organelle) of the cell includes (e.g., expressed in) a second TK protein including a second TK self complementing fragment. The second TK self complementing fragment includes a second portion (e.g., N-terminal fragment of SEQ ID No: 1) of the TK protein that is complementary with the first TK self complementing fragment. The first TK self complementing fragment and the second TK self complementing fragment are not active, as described herein. When brought into contact with one another, the first TK self complementing fragment and the second TK self complementing fragment spontaneously self complement to substantially form the active TK protein. The active TK protein can interact with a labeled TK substrate. The labeled TK substrate is modified (e.g., phosphorylated) and can accumulate in the cell, for example. The modified labeled TK substrate(s) can produce a signal and subsequently be detected. Therefore, the cellular localization of the target protein can be detected, studied, monitored, and/or evaluated.

In particular, embodiments of the present disclosure include imaging protein-protein interactions in living subjects using imaging compounds of the present disclosure. Embodiments of the present disclosure include the molecular engineering rationale and construction of positron emission tomography (PET)-based reporters (the herpes simplex virus type 1 thymidine kinase [HSV1-TK or TK]) split into two fragments between Thr-265 and Ala-266 (SEQ ID No: 1, 1 to 265, and 266 to 376, respectively).

In addition, embodiments of the disclosure include the use of the split TK fragments in a protein fragment-assisted complementation assay (PCA) to quantitatively measure real time protein-protein interactions in mammalian cells using cell uptake studies, and to image these with microPET in a subcutaneous xenograft model in living mice.

Furthermore, an embodiment of the present disclosure includes the introduction of a point mutation (V119C in SEQ ID No: 1, amino acids 1 to 265) in the N-terminal fragment of the split TK reporter to significantly enhance the level of protein-protein interaction-induced reporter complementation. The designing of this split TK reporter and its application in an in vivo PET-based PCA allows for more precise fully quantitative and tomographic localization of protein-protein interactions in pre-clinical small and large animal models of disease than has been possible previously. Embodiments of the present disclosure enable deep tissue imaging. Additional details regarding the imaging compound are described in Example 1.

Briefly described, embodiments of this disclosure, among others, include split protein fragments, split protein fragments systems, fusion proteins including split protein fragments, vectors encoding split protein fragments, and methods of using the split protein fragments, fusion proteins, vectors, and the like. Note that for each protein, fusion protein, protein fragment, and nucleotide, one skilled in the art would be able to determine the corresponding nucleotide sequence or protein sequence, respectively.

The split protein fragments can be included in a fusion protein. For example, the fusion protein can include the split protein of one of the fragments while also including a protein of interest and/or other proteins, linker, and/or other components consistent with the teachings of this disclosure. The split protein fragments or a fusion protein including the split protein fragment can be expressed in a system (e.g., a cell) using a vector, for example, as known in the art.

Each of the fragments or the fusion protein vectors can include, but are not limited to, polynucleotides that encode the fragments of the present disclosure as described above and degenerate nucleotide sequences thereof. Methods of producing vectors are well known in the art.

### First and Second Self Complementing TK Fragments

As noted above, the first TK self complementing fragment is associated (e.g., biologically (e.g., bound to or expressed in), chemically (e.g., ionic bond, covalent bond, hydrogen bonding, and the like), and/or physically) to a first target or system (e.g., a protein, a peptide, a cell (e.g., inside of or outside of), an organelle, a drug, a macromolecule, and the like). In an embodiment, the first TK self complementing fragment can be linked to the first target or system via a linking compound or peptide.

As noted above, the second TK self complementing fragment is associated (e.g., biologically (e.g., bound to or expressed in), chemically (e.g., ionic bond, covalent bond, hydrogen bonding, and the like), and/or physically) to a second target or system (e.g., a protein, a peptide, a cell (e.g., inside of or outside of), an organelle, a drug, a macromolecule, and the like). The second TK self complementing fragment can be linked to the second target or system via a linking compound or peptide (e.g., TAT, or combinations thereof).

The first TK self complementing fragment can be selected from the N-terminal fragment of the TK protein (SEQ ID No: 1, amino acids 1-265) or the C-terminal fragment of the TK protein (SEQ ID No: 1, amino acids 266-376). The first TK self complementing fragment and the second TK self complementing fragment are not the same. In this regard, the second TK self complementing fragment can be the other of the N-terminal fragment of the TK protein (SEQ ID No: 1, amino acids 1-265) or the C-terminal fragment of the TK protein (SEQ ID No: 1, amino acids 266-376). In an embodiment, one of the first and second TK self complementing fragment can be a modified N-terminal fragment of the TK protein (V119C in SEQ ID No: 1, amino acids 1 to 265), while the other is the C-terminal fragment of the TK protein.

The TK protein or the split protein TK self complementing fragments can include conservatively modified variants as long as the conservatively modified variant retains the characteristics of the TK protein (e.g., able to interact with the TK substrates to modify them via phosphorylation or the like) or the split protein self complementing fragments. It should be noted that polynucleotides encoding the conservatively modified variants are intended to be disclosed by this disclosure. Additional details concerning the TK protein and self complementing fragments thereof are described in the Examples below.

The split protein TK self complementing fragments can be included in a protein such as a fusion protein. For example, the fusion protein can include the split protein of one of the self complementing fragments, while also including a protein of interest and/or other proteins, linker, and/or other components consistent with the teachings of this disclosure. The split protein TK self complementing fragments or a protein including the split protein self complementing fragment can be expressed in a system (e.g., a cell) using a vector or other expression system or method, for example.

Note that for each protein, fusion protein, protein fragment, and nucleotide, one skilled in the art would be able to determine the corresponding nucleotide sequence or protein sequence, respectively. In addition, methods known in the art can be used to produce proteins, fusion proteins, protein fragments, nucleotides, vectors, and the like.

### TK Fragment Vector(s) and Expression System(s)

Embodiments of the present disclosure include, but are not limited to, polynucleotides that encode the first TK self complementing fragment, the second TK self complementing fragment, first TK protein, second TK protein, and the like, as described above and degenerate nucleotide sequences thereof, as well as fusion proteins of the first TK self complementing fragment, the second TK self complementing fragment, first TK protein, second TK protein, and the like, and degenerate nucleotide sequences thereof. Methods of producing vectors, other expression systems, (e.g., viral and non-viral), and polynucleotides are well known in the art. It should be noted that the fusion protein can be expressed using other expression systems, and the vector is merely an illustrative embodiment.

### Labeled TK Substrates

The labeled TK substrates can include compounds or proteins that interact with the active TK protein and can be subsequently detected. In an embodiment, the labeled TK substrate can include, but is not limited to, uracil nucleoside derivatives labeled with radioactive iodine (e.g., FIAU or radiolabeled 2′-fluoro-2′-deoxyarabinofuranosyl-5-ethyluracil (FEAU)), and/or acycloguanosine derivatives labeled with radioactive ¹⁸F-Fluorine (e.g., fluoropenciclovir [FPCV] or 9-(4-[¹⁸F]-fluoro-3-hydroxymethylbutyl)-guanine [FHBG]).

It should be noted that the label on the labeled TK substrate can be selected from labels such as, but not limited to, F-19 (F-18), C-12 (C-11), I-127 (I-125, I-124, I-131, I-123), Cl-36 (Cl-32, Cl-33, Cl-34), Br-80 (Br-74, Br-75, Br-76, Br-77, Br-78), Re-185/187 (Re-186, Re-188), Y-89 (Y-90, Y-86), Lu-177, and Sm-153. It should be noted that an alternative way to represent F-18, C-11, and the like, is the following: ¹⁸F and ¹¹C respectively, and both ways are used herein. Preferred labeled TK substrates of the present disclosure are labeled with one or more radioisotopes including ¹¹C, ¹⁸F, ⁷⁶Br, ¹²³I, ¹²⁴I, or ¹³¹I and in an embodiment the labels are ¹⁸F, ⁷⁶Br, or ¹²³I, ¹²⁴I or ¹³¹I and are suitable for use in peripheral medical facilities and PET clinics.

In an embodiment of the present disclosure, the label can be a SPECT isotope. The SPECT isotope can include, but is not limited to, ¹²³I, ¹²⁵I, ¹³¹I, ⁹⁹Tc, ¹¹¹In, and ^{186/188}Re and combinations thereof, as well as those described in the figures.

The imaging systems can include, but are not limited to, optical systems, magnetic systems, x-ray systems, nuclear systems, positron emission tomography (PET) imaging systems, ultrasound systems, and the like. In particular, the imaging techniques can include, but are not limited to, NIR fluorescence, intravital microscopy, X-ray computed tomography (CT), magnetic resonance imaging (MRI), ultrasound (ULT), single photon emission computed tomography (SPECT), PET, and combinations thereof. In an embodiment, PET imaging is a preferred embodiment.

As mentioned above and described in more detail below and in the Examples, embodiments of the split protein system can be used to image, detect, study, monitor, evaluate, and/or screen, protein-protein interactions, diseases, conditions, and related biological events in vivo or in vitro.

### Target(s)

The first target and the second target can each be selected from, but are not limited to, a specific protein, a cell type, a receptor, a transporter, an antibody, an antigen, a polypeptide, an aptamer, a small molecule, and a saccharide (e.g., a monosaccharide, a disaccharide and a polysaccharide). In an embodiment, the first and second targets are selected to determine protein-protein interactions. For example, in an embodiment the first and second targets can be: FRB interacting with FKBP12 in the presence of Rapamycin, HIF-1alpha interacting with pVHL, or antiparallel leucine zipper peptides interacting with each other.

### Linker Compound or Peptide

As noted above, the first and/or second TK proteins can include one or more linkers between or among the TK self complementing fragments and/or targets or systems. In an embodiment, the linker is a peptide that can be a peptide that bonds directly or indirectly to the components of the first and/or second TK proteins.

The first linker peptide and/or other linker peptides selected for an embodiment may depend at least upon the strength of the complementation potential between the first split TK protein fragment and the second split TK protein fragment, and the like. Table 1 illustrates some exemplary peptide linkers.

TABLE 1
illustrates exemplary linkers.

| | Amino acid sequence of |
|---|---|
| SEQ. ID No. | illustrative linkers |
| #3 | GGGGSGGGGS |
| #4 | ACGSLSCGSF |
| #5 | EAAAREAAAR |
| #6 | EAAAREAAAREAAAREAAAR |
| #7 | ACGSLSCGSFACGSLSCGSF |
| #8 | ATSATATSAT |

### Methods of Use

Embodiments of this disclosure include, but are not limited to, methods of imaging tissue or a host using an embodiment of the split protein TK fragments, proteins, and systems. Embodiments of the present disclosure can be used to image, detect, study, monitor, evaluate, and/or screen, protein-protein interactions in vivo or in vitro using embodiments of the present disclosure. In particular, embodiments of the present disclosure include methods of imaging protein-protein interactions and methods of cellular localization of proteins. In an embodiment, living hosts can be imaged.

In general, embodiments of the split protein TK fragments, proteins, and systems can be used in imaging protein-protein interactions. For example, the split protein TK fragments, proteins, and/or systems is provided or administered to a host in an amount effective to result in uptake of the imaging compound into the host. Additional details are described above. The host is then introduced to an appropriate imaging system (e.g., PET system) for a certain amount of time. The labeled TK substrate could be detected using the imaging system. Additional information regarding the first fragment and the second fragment of the split protein system are described in Example 1.

It should be noted that the amount effective to result in uptake of the split protein TK fragments, proteins, and/or systems into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

As mentioned above, typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood urine or tissue samples of the animals mentioned for veterinary applications.

### Kits

The present disclosure also provides split protein TK fragments, proteins, and/or systems. In an embodiment, the kit may include a pharmaceutically acceptable carrier and split protein TK fragments, proteins, and/or systems of the disclosure. In certain embodiments, the packaged pharmaceutical composition includes the reaction precursors to be used to generate the split protein TK fragments, proteins, and/or systems according to the present disclosure. The kits may further include indicia including at least one of: instructions for using the split protein TK fragments, proteins, and/or systems to image a host, or host samples (e.g., cells or tissues), which can be used to image protein-protein interactions, for example.

This disclosure encompasses kits that include, but are not limited to, split protein TK fragments, proteins, and/or systems and directions (written instructions for their use). The components listed above can be tailored to the particular biological event (e.g., protein-protein interaction) to be monitored as described herein. The kit can further include appropriate buffers and reagents known in the art for administering various combinations of the components listed above to the host cell or host organism. The split protein TK fragments, proteins, and/or systems and carrier may be provided in solution or in lyophilized form. When the split protein TK fragments, proteins, and/or systems and carrier of the kit are in lyophilized form, the kit may optionally contain a sterile and physiologically acceptable reconstitution medium such as water, saline, buffered saline, and the like.

### EXAMPLES

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### INTRODUCTION

There is a pressing need to develop better techniques for noninvasive imaging of protein-protein interactions (PPIs). We describe the molecular engineering of a novel positron emission tomography (PET)-based reporter (the herpes simplex virus type 1 thymidine kinase [TK]) split between Thr-265 and Ala-266. We used this reporter in a protein fragment-assisted complementation assay (PCA) to quantitatively measure PPIs in mammalian cells using cell uptake studies, and to image these interactions with microPET in living mice. We also introduced a point mutation (V119C) in the N-terminal fragment of split TK to significantly enhance the level of PPI-induced reporter complementation. We observed significant TK complementation in a PCA based on rapamycin modulation of FRB and FKBP12, and in an intramolecular protein folding assay based on the estrogen receptor. The designing of this novel split TK reporter and its application in an in vivo PET-based PCA potentially allows for more precise fully quantitative and tomographic localization of PPIs in pre-clinical small and large animal models of disease than has been possible to date. It should also be noted that the novel split TK reporter approach described here has the potential for the first time to provide a sensitive and more accurate means of in vivo fully quantitative imaging and precise tomographic localization of protein-protein interactions deeper in the body than is currently possible with available optical imaging techniques, which, by contrast, and rather restrictingly, are relatively surface weighted and semi-quantitative in nature. This innovation may result in a significant benefit when attempting accurate study of more representative animal models of disease based on orthotopic cellular implants, and more so in transgenic animal applications during the pre-clinical drug discovery and validation process.

### Results:

### Design of the Split TK PCA Strategy

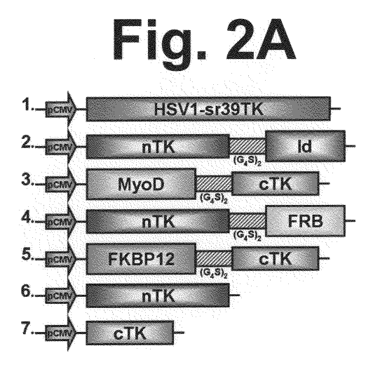

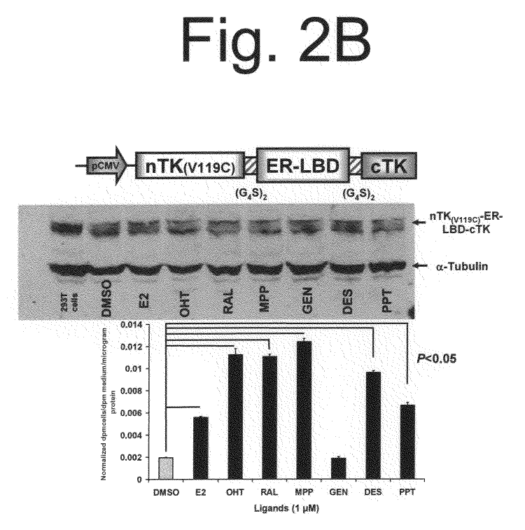

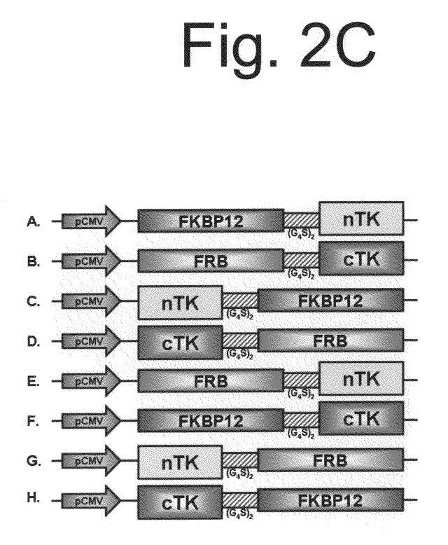

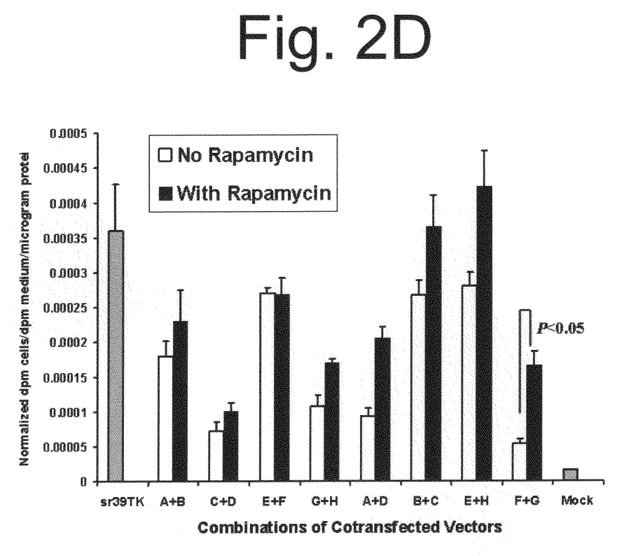

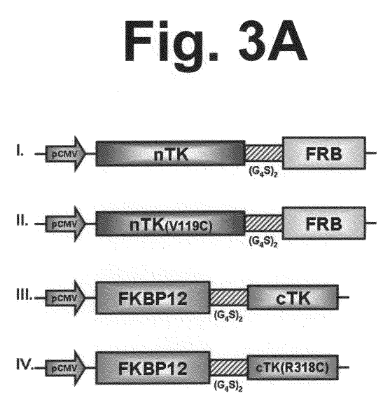

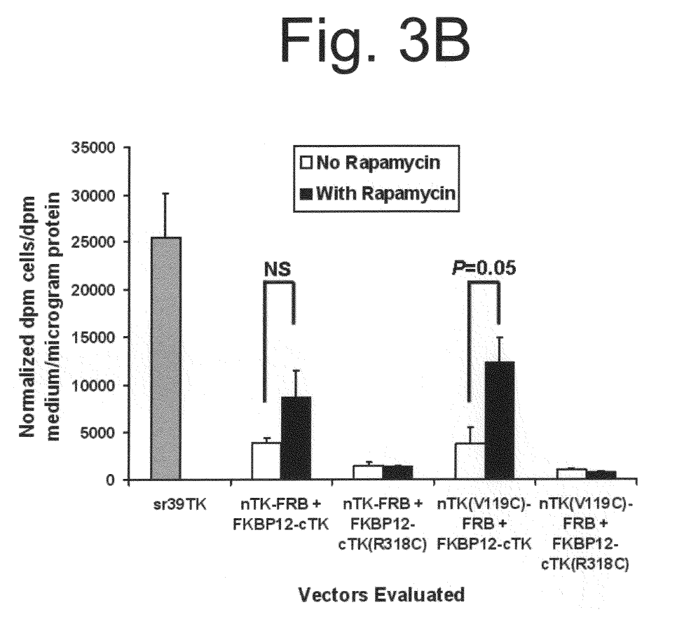

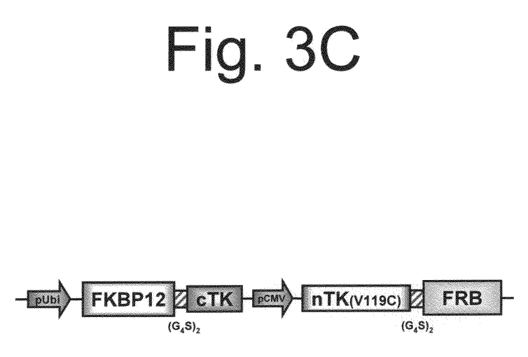

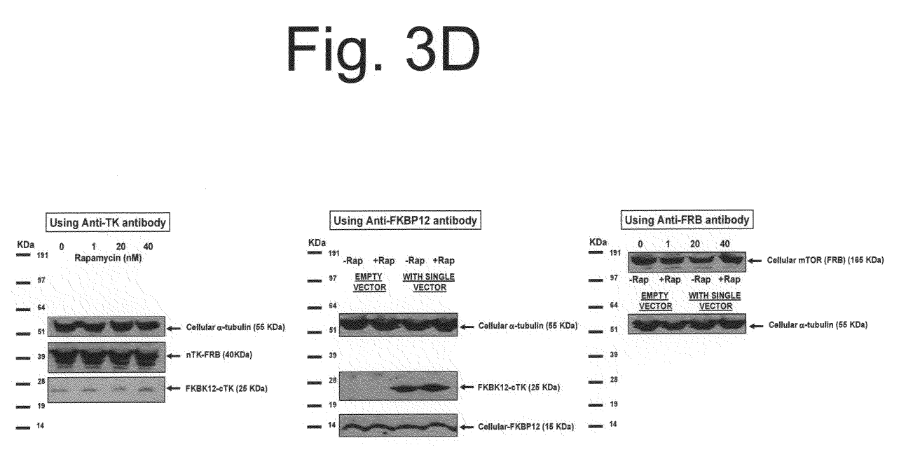

Following a partial circular permutation screen we deemed it likely that a split in the TK reporter protein between Thr-265 and Ala-266 would produce two protein fragments that could be further tested in a PCA strategy for imaging protein-protein interactions (Supplementary Discussion and FIG. 3). We next determined if removal of the linker joining the native N- and C-terminal ends in cpTK₂₆₅ (circular permuted TK split at residues 265/266) would yield a pair of TK fragments that could function and complement in a PCA strategy (Proc. Natl. Acad. Sci. USA 99, 15142-15147 (2002), which is incorporated herein by reference regarding the material related to this discussion). We used the two heterologous human proteins FRB and FKBP12 that are known to strongly interact in the presence of rapamycin (Proc. Natl. Acad. Sci. USA 92, 4947-4951 (1995), which is incorporated herein by reference regarding the material related to this discussion). The use of the FRB/FKBP12/rapamycin system is an example of a “three-hybrid” interaction in which a third partner (e.g., a small ligand, rapamycin in this case) mediates a protein-protein interaction (Proc. Natl. Acad. Sci. USA 96, 5394-5399 (1999) and Nat. Biotechnol. 17, 683-690 (1999), each of which is incorporated herein by reference regarding the material related to this discussion). The value of this test system has been demonstrated in several previous PCA strategies, including the DHFR fragment complementation assay, and those using split Firefly luciferase and Renilla luciferase. The N-terminal TK fragment comprising the amino acids Met-1 to Thr-265 (nTK) was fused (via an intervening flexible 10-residue linker [GGGGS]₂) proximal to the N-terminal end of FRB, to yield the chimeric protein nTK-FRB. The C-terminal TK fragment comprising the amino acids Ala-266 to Asn-376 (cTK) was fused (via a similar linker) distal to the C-terminal end of FKBP12, to yield the chimeric protein FKBP12-cTK (FIG. 2a). We initially used this particular orientation because it was successful in previously published PCA strategies, e.g., when using split Firefly luciferase, and Renilla luciferase, both when used with the same FRB/FKBP12 heterodimerization system. We tested the interaction of these proteins (FRB and FKBP12, modulated by rapamycin) upon transient co-expression of the fusion proteins in 293T cells where there is no endogenous HSV1-sr39tk expression, followed by an in vitro TK enzyme cellular uptake assay to detect dimerization-assisted complementation of the TK fragments (see results in next section).

To demonstrate further general applicability of the engineered split TK fragments we also tested their complementation in a separate system that employs an intramolecular protein folding sensor. We designed and validated this system previously by encoding various human estrogen receptor ligand-binding domain (hER-LBD) fusion proteins that could lead to split reporter complementation in the presence of the appropriate ligands (Proc. Natl. Acad. Sci. USA 103, 15883-15888 (2006), which is incorporated herein by reference regarding the material related to this discussion). For this, we constructed a vector that transiently expresses a fusion protein with split TK fragments (containing a V119C point mutation, see below) and hER-LBD (FIG. 2b), and studied this in 293T cells after treatment with several estrogen receptor ligands. There was a significant level of ligand-induced split TK complementation for several ligands (FIG. 2b), in a similar pattern to that documented previously for complementation of split synthetic Renilla luciferase complementation (Proc. Natl. Acad. Sci. USA 103, 15883-15888 (2006), which is incorporated herein by reference regarding the material related to this discussion). All experiments were conducted with comparison to a positive control derived from the activity of intact TK, and a mock transfection acting as a negative control.

### Orientation of Fusion Proteins for Complementation of Split TK Fragments

We next studied if the construction of the chimeric proteins nTK-FRB and FKBP12-cTK, used to this point in testing complementation of split TK fragments, did indeed represent the optimal orientation of heterodimeric interacting proteins relative to attached split TK fragments. A consideration regarding designing these chimeras is how can one fuse the fragments of a split reporter to the interacting proteins, and specifically, what choices of N- or C-termini to use for this purpose.

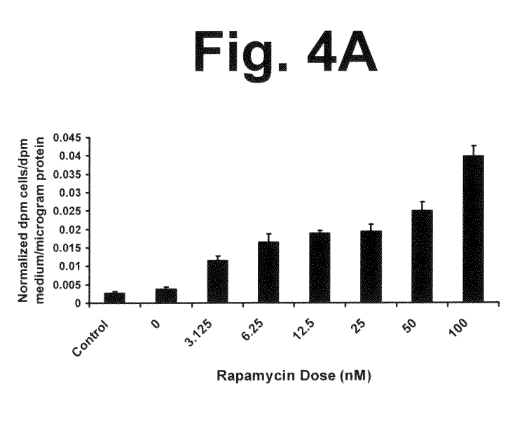

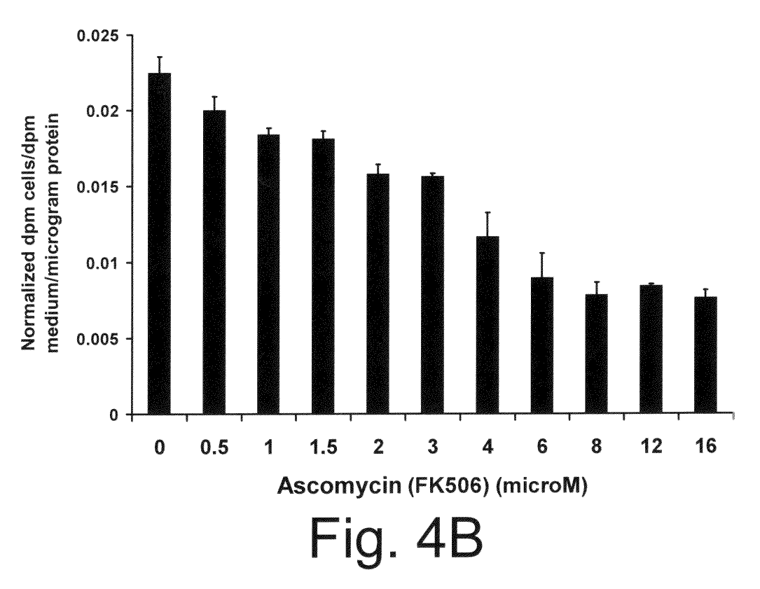

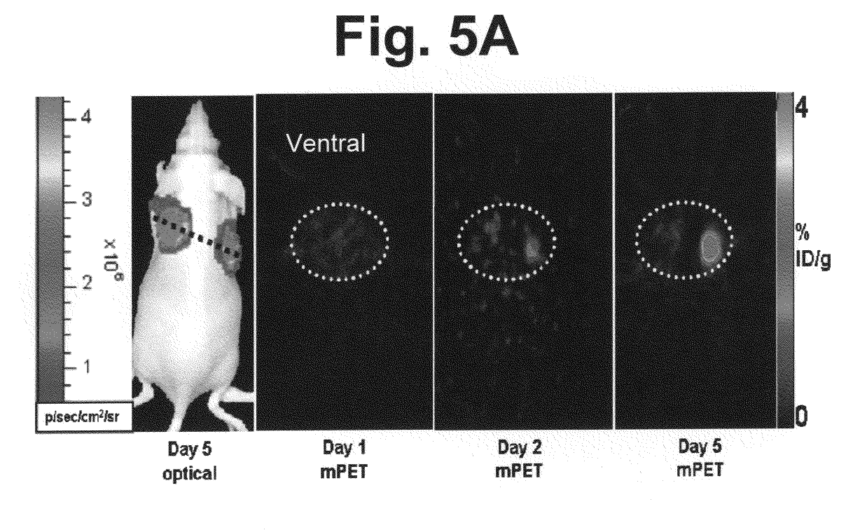

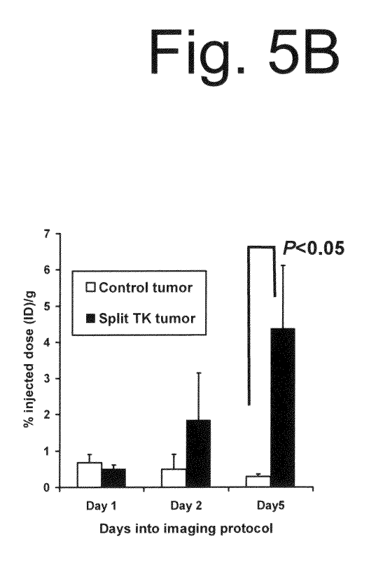

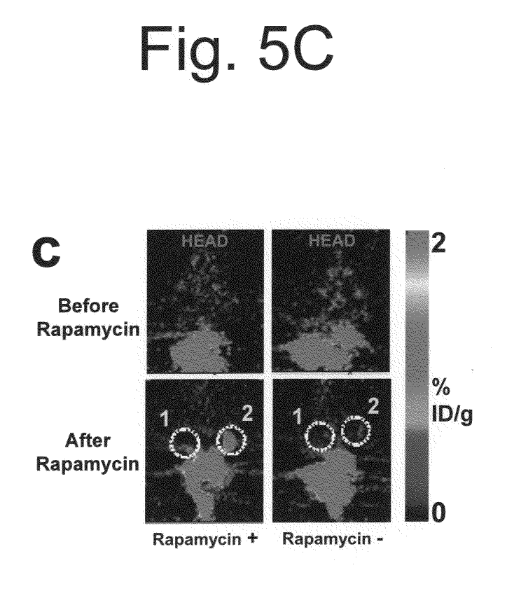

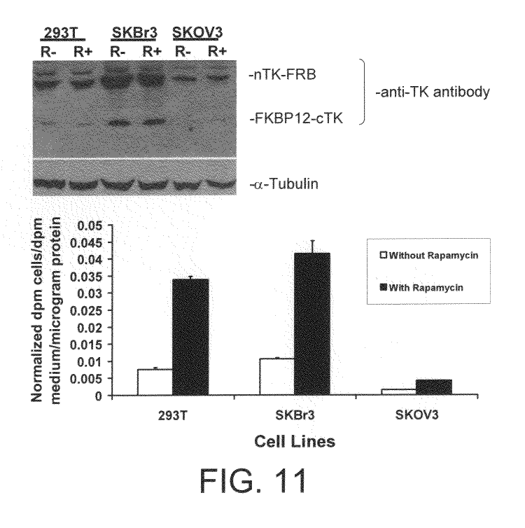

To evaluate the interaction of any two given chimeric proteins, we constructed all eight possible chimeras with upstream and downstream orientations of nTK or cTK relative to FRB or FKBP12 (FIG. 2c). This allowed a study of all eight combinations of interacting protein chimeras upon co-transfection of constructs A and B, C and D, E and F, G and H, A and D, B and C, E and H, and F and G, as shown in (FIG. 2c). The optimal combination was found to be nTK-FRB interacting with FKBP12-cTK (combination F+G) (FIG. 2d), precisely the orientation chosen at the outset of the study. We deemed this one of the best of the eight combinations because it yielded the lowest level of complemented TK in the absence of rapamycin (14.8% of the enzyme activity of full length TK, but also 2.3-fold above mock transfection levels), and the largest relative gain (an ˜3-fold induction, P<0.05) in complemented TK activity after rapamycin administration (amounting to 46.1% of the enzyme activity of full length TK). Low restored enzyme activity levels in the absence of rapamycin and large gains after rapamycin would constitute desirable features for optimal functioning of this PCA strategy (see Discussion). When tested in 293T cells other combinations resulted in higher absolute levels of complemented TK activity, but considerably elevated levels (above mock levels) were also present prior to addition of rapamycin. Using the determined combination of optimally oriented chimeras we further studied the temporal changes in split TK complementation over the first 60 hours after transient transfection (Supplementary Discussion and FIG. 4). We also determined a suitable dose of rapamycin (40 nM per well in a 12-well plate) to study this complementation process (Supplementary Discussion and FIG. 5). In addition, we tested the combination of optimally oriented chimeras in two other cell lines (SKBr3 human breast cancer, and SKOV3 human ovarian cancer), which yielded a similar approximate 3-fold rise in split TK complementation upon protein-protein interaction (FIG. 11).

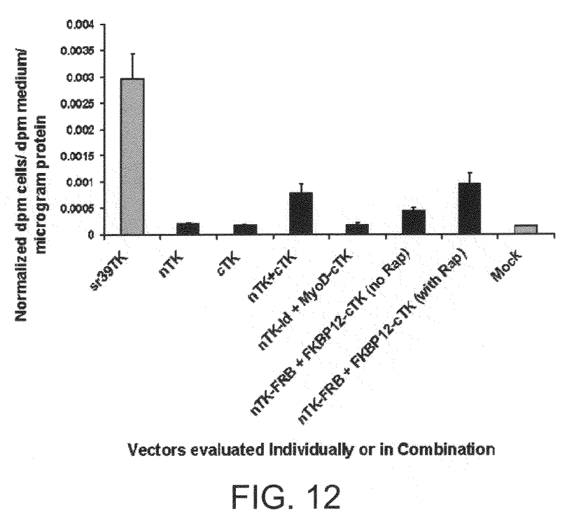

We separately evaluated in detail the potential presence of self-complementation of split TK fragments, and whether the interacting proteins themselves might sterically hinder complementation of attached split TK fragments. These findings are presented in the Supplementary Discussion and FIGS. 11 and 12.

### TK Fragment Point Mutations to Optimize the Split TK PCA Strategy

We next investigated the possibility of diminishing self-complementation and augmenting protein-protein interaction-induced complementation of split TK fragments by introducing two point mutations in the TK molecule. Degreve et al. have shown that the Arg-318 residue of TK is especially important for homodimerization (Biochem. Biophys. Res. Comm. 264, 338-342 (1999), which is incorporated herein by reference regarding the material related to this discussion). R318 hydrogen bonds with a water molecule, which in turn hydrogen bonds with the main chain carbonyl of the alanine residue at position 137 of the opposite monomer. Mutation of R318 leads to disruption of the interactions that stabilize the dimer and yields a predominant population of TK monomers. Arginine is a hydrophilic amino acid; we therefore point mutated this to a hydrophobic cysteine residue, yielding R318c. On the other hand, Wurth et al. have shown that the Val-119 residue of TK, one in each subunit of the dimer, and located in the NMP-binding domains, appear to be sufficiently close to each other to permit disulfide bond formation when mutated into cysteines (J. Mol. Biol. 313, 657-670 (2001), which is incorporated herein by reference regarding the material related to this discussion). This crosslinks the two monomers covalently, resulting in a mutant with properties identical to the original TK enzyme. We therefore point mutated V119 to a cysteine residue, yielding V119C. The nTK fragment containing the mutation V119C (nTK_{(V119C)}) was fused upstream of FRB, and cTK containing the mutation R318c (cTK_{(R318C)}) was fused downstream of FKBP12 (FIG. 3a).

We next tested the interaction of FRB and FKBP12 upon transient co-expression of the appropriate pairs of fusion proteins from the available choices of nTK-FRB, FKBP12-cTK, nTK_{(V119C)}-FRB, and FKBP12-cTK_{(R318C)} in 293T cells, followed by an in vitro TK enzyme cellular uptake assay to detect dimerization-assisted complementation of the TK fragments. The results indicated that no restored TK activity was detected whenever cTK_{(R318C)} was one of the split reporter fragments (FIG. 3b). This mutation seemed to prevent both self-complementation and protein interaction-assisted complementation, even in the presence of nTK_{(V119C)}, suggesting perhaps that the R318c mutation might have a dominant functional effect (TK monomerization) over the dimerizing effect of V119C. Alternatively, and as alluded to by Degreve et al. in a different context, R318c might result in an overall conformational change of the TK protein that, in the context of this study, might render it inactive. On the other hand, when nTK_{(V119C)} was co-expressed with FKBP12-cTK (that is, in the absence of the R318c mutation), there was a prominent (41%) rise in the protein interaction-assisted level of TK complementation, consistently observed with repeated transient transfection experiments. This noticeable rise was at the limit of statistical significance (P=0.05) when compared with the PCA without the V119C mutation. Self-complementation levels, however, still remained similar to those seen after co-expression of the standard nTK-FRB and FKBP12-cTK fusion proteins.

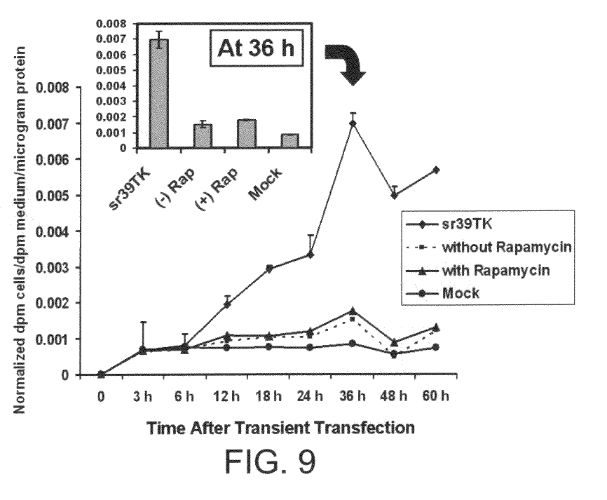

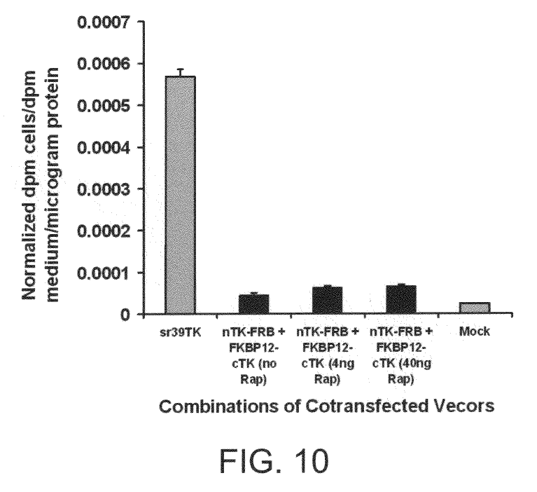

We also studied the reversibility of our point-mutated split TK-based PCA using a ligand-reversible dimer of a mutant FKBP12 called F_{M} (F36M) that can be disrupted by FK506 (Proc. Natl. Acad. Sci. USA 103, 15883-15888 (2006), which is incorporated herein by reference regarding the material related to this discussion). Our results confirm that disassembly of the folded TK reporter is possible even after split TK fragments have complemented following a protein-protein interaction (Supplementary Discussion and FIGS. 9 and 10).

### Characterization of 293T Stable Cells

We prepared 293T cells stably expressing nTK_{(V119C)}-FRB and FKBP12-cTK in a single vector (FIG. 3c) and tested them for the expression of split TK plus the interacting proteins using the in vitro [8-³H]Penciclovir cell uptake assay before and after 36 h exposure of cells to rapamycin. Expression levels of FRB and FKBP12 (in their fusion form or endogenous to these stable cell) were determined in total cell lysates by immunoblotting with the corresponding antibodies, and were found to be adequate for use of these protein chimeras in a PCA strategy. Various doses of rapamycin (a known cell-cycle inhibitor at high concentrations) up to the standard 40 nM dose used in this study had no effect on expression levels of these chimeras (FIG. 3d).

The cells selected and subsequently used for imaging also responded in a dose dependent manner to increasing rapamycin doses up to 100 nM when tested in a cell-culture rapamycin escalation dose study (FIG. 4a). We further scrutinized the association of the rapamycin-FKBP12 complex with FRB because, should it be possible to demonstrate that the formation of this complex is directly induced by rapamycin and competitively inhibited by ascomycin (FK506), this would confirm that the observed complementation of split TK is driven by a specific molecular interaction (Nat. Methods 3, 977-979 (2006), which is incorporated herein by reference regarding the material related to this discussion). Since ascomycin competes with rapamycin for binding to FKBP12, we studied this competitive binding in these stably transfected 293T cells by adding escalating concentrations (from 0 to 16 μM) of ascomycin along with 40 nM rapamycin 36 h prior to in vitro [8-³H]Penciclovir cell uptake assay. The results showed that increasing the concentration of ascomycin led to a reduction in TK complementation (FIG. 4b), likely because it blocked the binding of rapamycin to FKBP12 and thus reduced the heterodimerization with FRB. Given the particular increments we used in this study, the smallest concentration of ascomycin required to initiate the competitive inhibition of rapamycin was found to be 0.5 μM.

### MicroPET Imaging of Protein-Protein Interactions in Subcutaneous Xenograft Tumor Models in Mice

Next, 5 mice subcutaneously injected with these 293T cells stably expressing nTK_{(V119C)}-FRB plus FKBP12-cTK from a single vector were imaged using the [¹⁸F]-FHBG probe and microPET in the presence of rapamycin. The signal from each implant was quantified directly from the microPET images to determine the % ID/g for the FHBG probe. These 5 mice were allowed initially to grow 1-wk-old subcutaneous xenografts of 293T cells stably expressing nTK_{(V119C)}-FRB plus FKBP12-cTK on one shoulder and control tumors (mock transfected 293T cells) on the other shoulder prior to microPET imaging before and 24 h after exposure to 50 μg of rapamycin. The animals were repeatedly imaged on day 1 (before rapamycin), day 2 (one dose post rapamycin) and day 5 (3 doses post rapamycin) of this imaging protocol, i.e. after 1 week of initial xenograft growth. The incremental increases in mean % ID/g values for FHBG accumulation in the tumor formed by cells stably expressing nTK_{(V119C)}-FRB plus FKBP12-cTK are shown in (FIG. 5a). Comparison was made with the control tumor showing background signal. Statistically significant differences in imaging signal were seen in tumors expressing split TK plus the interacting proteins when comparing probe accumulation pre- and post-injection of rapamycin (P=0.02 on imaging Day 5) (FIG. 5b). Only background signal was obtained from both implanted xenografts in a separate cohort of 4 control mice not receiving rapamycin (data not shown). In another group of 4 mice, xenografts expressing nTK_{(V119C)}-FRB plus FKBP12-cTK were compared with control implants stably expressing nTK plus cTK only (also in a single vector). There was minimal FHBG accumulation in the control tumors on the fifth day of a similar imaging protocol (FIG. 5c). Preliminary pilot imaging studies using transiently transfected cells had also been carried out in 3 mice using C6 cells stably transfected with HSV1-sr39tk as positive controls (data not shown).

As anticipated, the microPET detected counts from shoulder tumors expressing split TK plus interacting proteins did not differ in significance whether the animals were imaged in the supine or prone positions (probe % ID/g of 0.89 and 1.02 respectively, Supplementary FIG. 12). The optical cooled charge-coupled device (CCD) camera bioluminescence imaging of the co-injected 293T cells stably expressing Firefly luciferase (see Methods) showed equivalent signal from both control and experimental tumors in all the animals used for microPET imaging, reflecting on the equivalent cell load and viability in all tumors under study (FIG. 5a). However, these optical imaging findings were evident only upon imaging the mice in the prone position, with a peak light emission of 2.3×10⁵ p/sec/cm²/sr (Supplementary FIG. 12). In contrast, when imaged in the supine position, the light emanating from subcutaneous xenografts on the shoulder/back did not penetrate through the full thickness of the mouse (the mouse torso being >1 cm thick) (Supplementary FIG. 12). These findings highlight the relative advantage of PET over optical imaging in imaging sources of signal at depths greater than 1 cm from the exterior which is a well known advantage of PET over optical bioluminescence imaging.

### DISCUSSION

Typical protein-protein interactions represent low level biological events, and are thus challenging to locate and image in intact living subjects. Therefore, members of our group and others have recently directed considerable efforts toward exploiting the inherent high sensitivity (thought to be in the 10⁻¹⁵ to 10⁻¹⁷ mole/L range¹¹) of optical bioluminescence imaging using cooled CCD cameras to image and quantify in living mice very low levels of visible light that mirror typical protein-protein interaction events (Proc. Natl. Acad. Sci. USA 99, 377-382 (2002) and Annu. Rev. Biomed. Eng. 4, 235-260 (2002), each of which is incorporated herein by reference regarding the material related to this discussion). As such, we previously reported an inducible yeast two-hybrid system with Firefly luciferase, and a split Firefly luciferase PCA to study protein-protein interactions in cell lines, and to non-invasively image interactions in living mice (Proc. Natl. Acad. Sci. USA 99, 3105-3110 (2002) and Proc. Natl. Acad. Sci. USA 99, 15608-15613 (2002), each of which is incorporated herein by reference regarding the material related to this discussion). For similar reasons, we more recently developed a bioluminescence resonance energy transfer BRET2 system that uses Renilla luciferase (hRluc) protein and its substrate DeepBlueC as an energy donor, and a mutant green fluorescent protein (GFP²) as the acceptor (FASEB J. 19, 2017-2019 (2005), which is incorporated herein by reference regarding the material related to this discussion). Moreover, we devised an inducible split hRluc PCA bioluminescence assay to quantitatively measure real time protein-protein interactions in mammalian cells and to image these in living mice in the presence of the substrate coelenterazine (Anal. Chem. 75, 1584-1589 (2003), which is incorporated herein by reference regarding the material related to this discussion). We, and others, previously reviewed these various techniques available for molecular imaging of protein-protein interactions in living subjects (Trends. Anal. Chem. 24, 446-458 (2005), Curr. Opin. Biotechnol. 18, 31-37 (2007), Methods 29, 110-122 (2003), Chem. Rec. 3, 22-28 (2003), and Anal Chim Acta 556, 58-68 (2006), each of which is incorporated herein by reference regarding the material related to this discussion).

The overall advantages of noninvasive diagnostic molecular imaging technologies (such as the ability to assess whole body phenomena, repeatability, functionality, and quantification) could be made more apparent with even greater exploitation of the benefits and accuracy of each imaging modality. Unfortunately, significant limitations of optical bioluminescence imaging arise on account of the considerable attenuation of light in tissues deeper than 1 cm from the exterior (e.g., skin, mucosa, or an exteriorized surgical bed) and the lack of fully quantitative and truly tomographic capabilities. Moreover, the inability to compare signal from different locations owing to variable delivery of substrate, especially on occasion with coelenterazine, contributes further to its semi-quantitative nature. These relative concerns are also to be reckoned with the absence of an equivalent imaging modality readily applicable to human imaging studies, thus preventing potential future direct translation of developed experimental methods in mice for clinical use. There is therefore an urgent need to develop inherently more precise and quantitative noninvasive techniques that could be used to image protein-protein interactions deep within experimental small and large animals.

One such imaging modality is PET, which also has a relatively high sensitivity, in the range of 10⁻¹¹ to 10⁻¹² moles/L, mainly because it depends on coincidence detection of gamma rays for image generation, and attenuation factors can be precisely corrected in PET. The development of PET-based reporter gene expression imaging assays in the drug discovery and evaluation process is particularly advantageous because of the ability to validate them in cell culture, and, unlike optical imaging, to use them in a fully quantitative and truly tomographic manner in small animal models of disease (especially in transgenics). An added theoretical possibility might also include the future potential use of the same reporter probe in established clinical PET centers, assuming it would be possible to introduce reporter genes into humans as part of future gene and cellular therapies, or assuming intensive ongoing efforts to develop alternative simpler strategies for potential future human applications—such as the delivery of circulating exogenous split reporter proteins into cells using leader peptide sequences—do bear fruit. Indeed, the ability to perform translational research from a cell culture setting to pre-clinical animal models to clinical applications is one of the most unique and powerful features of PET technology. In part owing to its sensitivity and its fully quantitative and tomographic nature, PET is probably the only noninvasive imaging technology that currently can be applied in humans for in vivo monitoring of reporter gene expression, and with the potential to image complex biological phenomena such as protein-protein interactions.

An intact TK reporter had been used previously in a modified mammalian two-hybrid system to non-invasively PET image protein-protein interactions confined to the nucleus (Proc. Natl. Acad. Sci. USA 99, 6961-6966 (2002) and Cancer Res. 63, 1780-1788 (2003), each of which is incorporated herein by reference regarding the material related to this discussion). In this study we describe the molecular engineering rationale and construction of a novel split TK used in a PCA for in vivo PET molecular imaging of protein-protein interactions. This strategy is more advantageous, versatile, and with far greater potential applications than through use of a mammalian two-hybrid system in that it can image protein interactions throughout the cell and not just confined to interactions of nuclear proteins.

We studied all possible relative orientations of split TK and interacting protein fragments to determine the optimal orientation of chimeras for evaluation in this PCA assay. For the DHFR PCA strategy, Michnick et al. did not observe any difference in how the assays performed with diverse relative orientations of interacting proteins and attached split reporters, i.e. for DHFR the orientation of fusions was found to make little difference (Methods Enzymol. 328, 208-230 (2000) and Curr. Opin. Struct. Biol. 11, 472-477 (2001), each of which is incorporated herein by reference regarding the material related to this discussion). It was suggested that for proteins with clear domains that are contiguous with their polypeptide sequence, such as DHFR, it might be likely that any configuration would work, since the domain topology can be formed from any configuration of fusions. In contrast, however, when a protein does not have a domain structure that is contiguous with its sequence e.g. β-lactamase, and for single domain proteins, e.g., GFP, then, one configuration/orientation might be favored over any other. This was found to be the case also in bacterial PCA strategies based on aminoglycoside phosphotransferase and hygromycin B phosphotransferase.

The active site of TK includes the substrate nucleoside-binding pocket and the ATP nucleotide binding loop, as well as providing the residues that are responsible for coordinating the magnesium counterion. Thus, TK is a multi-domain protein with clear domains that are contiguous with its polypeptide sequence, but starting at residue Arg-46. Its domains are defined as CORE (the central 5 β-sheets: residues 46-81, 143-218, 227-250, 323-376), the NMP_{bind} domain (nucleoside monophosphate binding domain: residues 82-142), and the LID domain (peptide segment at the ATP-binding site: residues 219-226) (Protein Sci. 6, 2097-2106 (1997), which is incorporated herein by reference regarding the material related to this discussion). Therefore, although TK does possess clear domains that are contiguous with its polypeptide sequence, these domains are not present along the entire length of TK, but instead, are skewed away from the N-terminal of TK. Moreover, the two monomers align themselves in C₂ symmetry, a type of two-fold symmetry in which the units are related to one another by a two-fold axis (called a dyad axis), i.e. the two subunits are rotated 180 degrees from each other. Based on these two observations, we judged it likely that one configuration/orientation of interacting proteins relative to split reporters might be favored over any other for a PCA strategy based on split TK. Indeed, co-expression of nTK-FRB together with FKBP12-cTK gave the optimal orientation of chimeras in this PCA assay.

We introduced a point mutation (V119C) in TK to obtain a variant that permits disulfide bond formation and cross-linking of two TK monomers covalently. We showed that the use of nTK fragments carrying this point mutation resulted in 41% increased enhancement (which was at the limit of statistical significance) of the degree of protein-protein interaction induced complementation of TK fragments, as compared with use of non-mutated nTK (FIG. 3b), when studied 36 h post transient transfection in 293T cells. This, however, did not alter the degree of self-complementation of split TK fragments. The exact reasons for this noted rise in complemented TK activity when using nTK_{(V119C)} remain elusive. We know, for example, from the work of Wurth et al. that the cross-linked TK dimers have identical properties to TK in terms of expression yield, denaturing SDS PAGE gel electrophoresis, enzyme kinetics, CD spectra and thermal stability (J. Mol. Biol. 313, 657-670 (2001), which is incorporated herein by reference regarding the material related to this discussion). We also know that TK does not have to dimerize to become active; the dimer is enzymatically even reported to be less efficient than the monomer. Regardless of the precise mechanisms involved, we considered the consistent perceptible rise in the degree of protein-protein interaction-assisted complementation of TK fragments when using nTK_{(V119C)} to be useful, which potentially could be translated into a greater degree of imaging signal upon protein-protein interaction in a living subject than possibly seen with use of the non-mutated nTK fragment.

We used this mutated split TK in an in vivo PCA to quantitatively microPET image real time protein-protein interactions in 293T cells subcutaneously implanted in living mice. 293T cells were stably transfected with a single vector carrying nTK_{(V119C)}-FRB plus FKBP12-cTK and microPET imaging was performed after tail vein injection of the [¹⁸F]-FHBG probe. The signal from each implant (demonstrated to contain equal viable cell counts using independent optical imaging) was quantified directly from the microPET images to determine the % ID/g for the FHBG probe. Mock transfected cells acted as a negative control. The mean % ID/g for FHBG accumulation in the cells transfected with nTK_{(V119C)}-FRB plus FKBP12-cTK increased over 5 days of daily rapamycin administration to a value of 4.37±1.74, as compared to a background value of 0.28±0.10 for the mock transfected cells (FIGS. 5a and 5b). These results were statistically significant between experimental tumor and control groups (P=0.02), demonstrating for the first time the feasibility of protein-protein interaction PET reporter complementation imaging in living subjects using the molecularly engineered split TK system described herein.

In essence, the molecular engineering efforts employed in this study, like all protein engineering, are concerned with adapting proteins to function under different regimes (Nat. Rev. Mol. Cell. Biol. 3, 964-970 (2002), which is incorporated herein by reference regarding the material related to this discussion). In this regard, most protein engineering used so far in general, and in attempts to split TK more specifically, have been by ‘rational re-design’, i.e., preconceived alterations based on a detailed knowledge of protein structure, function and mechanism (Trends Biotechnol. 19, 13-14 (2001), which is incorporated herein by reference regarding the material related to this discussion). The alternative approach in enzyme engineering is through ‘directed evolution’, whereby libraries of mutated genes (normally by using error-prone PCR to create a library of mutagenized genes, but in this case the principle would be for these ‘mutations’ to be split genes that express fragments of TK) might be created, and genetic selection or high-throughput screening subsequently identifies the mutants (i.e., the split fragments) that possess retained TK activity. Although either rational re-design or directed evolution can be very effective, a combination of both strategies would probably represent the most successful route in future studies aimed at further refinement of the above strategies to obtain better functioning split TK fragments (exhibiting greater protein-protein interaction-induced complementation, with less self complementation) for use in a split reporter PCA strategy for PET imaging of protein-protein interactions.

Unlike fluorescence microscopy-based techniques, detailed studies of the kinetics of protein-protein interactions, including analysis of split reporter complementation reversibility, have been limited to date. More specifically, although it is possible in all molecular imaging split reporter complementation systems developed so far to record the occurrence of a protein-protein interaction event (i.e., a hypothetical switch from ‘off’ [no interaction] to ‘on’ [interaction]), a relative limitation of these systems had been the inability to determine the consequences of an event where two protein interacting partners to which split reporters are fused cease to interact (i.e., a switch back to ‘off’ from the ‘on’ position). Such a recognized limitation of PCAs has been recently addressed by Remy and Michnick (Nat. Methods 3, 977-979 (2006), which is incorporated herein by reference regarding the material related to this discussion) who demonstrated reversibility of a PCA based on split Gaussia luciferase using the ligand-reversible F_{M} system, also used in this study. We too demonstrated the ability of our complemented split TK fragments to exhibit natural reversibility when driven by disassembly of attached interacting proteins. This will be very useful in future imaging studies of drug induction and inhibition, as well as kinetic studies of protein complex assembly and disassembly with the split TK PCA. These future experiments will also require assessment in several more cell lines, as well as with a greater variety of protein partners of different sizes and interaction affinities (weak transient to strong obligate), to establish further general applicability of this technique.

These additional studies should also help to investigate the many other factors that could in theory have a bearing on data analysis and interpretation when imaging protein-protein interactions. These might include changes in the stability, solubility, enzymatic activity, and cellular localization of the complemented TK reporter.

### Methods

Details of the chemicals, enzymes, and reagents, as well as construction of all the plasmids and descriptions of cell culture and transfections can be found in Supplementary Methods, and elsewhere (Novel approaches to molecular imaging of protein-protein interactions in living subjects. PhD thesis, University of Cambridge (2007), which is incorporated herein by reference regarding the material related to this discussion). To assess the PCA strategy using split TK fragments upon heterodimerization of FRB and FKBP12 in 293T cells, 40 nM final concentration of rapamycin were added immediately after transfection. The cells were assayed after 36 hr incubation at 37° C. and in 5% CO₂.

### Cell Uptake Studies

Uptake of [8-³H]Penciclovir (0.76 μCi/ml, 1.5×10⁻⁵ mg/ml) was assessed 36 h after transient transfection of 293T cells. The cells were incubated at 37° C. for 4 hours. At the end of this period, radioactivity in the medium was measured. The wells were washed with 1×PBS (pH 7.2), the cells were harvested and the cell-associated radioactivity was determined with a Beckman LS-9000 Liquid Scintillation Counter with Biosafe II (Research products International) scintillation fluid, as described previously (Proc. Natl. Acad. Sci. USA 97, 2785-90 (2000), which is incorporated herein by reference regarding the material related to this discussion). Triplicate samples were evaluated for all uptake studies. The same wells were also used for determining total protein content²⁵. Data are expressed as the net accumulation of probe in [dpm cells/dpm medium (at start of exposure)/μg protein]±SE. For statistical analysis, the 2-tailed Student's t test was used. Differences were considered significant at P<0.05.

### Preparation and Testing of 293T Stable Cells

To make stable cells, we incubated 5×10⁶ 293T cells plated in a 10 cm dish for 24 h at 37° C. with 95% oxygen and 5% CO₂. The cells were then transfected with 10 μg of plasmid vector pcDNA-pUbi-FKBP12-cTK-pCMV-nTK_{(V119C)}-FRB using lipofectamine 2000 transfection reagent and 24 h later the medium was replaced with fresh medium containing 10 μg/ml puromycin. Every two days the medium was changed and replaced with fresh containing puromycin. The steps were repeated until we achieved 100% puromycin resistant cells. The cells were checked for the expression of split TK with the interacting proteins using the [8-³H]Penciclovir cell uptake assay before and after exposure of cells to 40 nM rapamycin. Details of a cell-culture rapamycin escalation dose study and an ascomycin competitive binding study on these cells, as well as Western analysis of protein expression levels are in Supplementary Methods.

### MicroPET Imaging in Living Mice

All animal handling and care was performed in accordance with Stanford University Animal Research Committee guidelines. For imaging studies using stable cell lines, five 12-week old female nude mice (nu/nu) were implanted subcutaneously over the left shoulder with 5×10⁶ mock transfected 293T cells admixed with 50,000 293T stable cells expressing Firefly luciferase (see below), and subcutaneously over the right shoulder with 5×10⁶ 293T stable cells expressing both nTK_{(V119C)}-FRB and FKBP12-cTK admixed with 50,000 293T stable cells expressing Firefly luciferase. The cells were allowed to grow as tumors for 7 days (to ˜0.3-0.4 cm in diameter). On day 8 mice were tail vein injected with 200 μCi of [¹⁸F]-FHBG after undergoing anesthesia with 2% isoflurane in oxygen at 2 L/min. Three hours later, mice were microPET imaged in a spread prone position in a FOCUS microPET scanner (Concorde Microsystems, Knoxville, Tenn.). Images were reconstructed using a three-dimensional filtered back projection and iterative maximum a posteriori algorithm, and no partial volume correction (Phys. Med. Biol. 43, 1001-1013 (1998), which is incorporated herein by reference regarding the material related to this discussion). Immediately after imaging the animals were intraperitoneally injected with 50 μg of rapamycin; 24 h later all the animals were re-subjected to microPET imaging as mentioned above. The animals were further injected with intraperitoneal 50 μg rapamycin for two more days at 24 h intervals and subjected to repeat microPET imaging. In a separate cohort of 4 control mice, the above temporal imaging studies were performed in an identical fashion except that no rapamycin was administered. The counts from regions of interest (ROI) were converted to the percentage injected dose per gram (% ID/g) of tumor using filtered back projection as described previously (Proc. Natl. Acad. Sci. USA 97, 2785-90 (2000), which is incorporated herein by reference regarding the material related to this discussion). This % ID/g is a measure of the amount of tracer accumulated in a given tissue site normalized to the injected amount and to the mass of the tissue examined. The FHBG accumulation (% ID/g) in the 293T tumor cells reflects their TK activity. For statistical analysis, the 2-tailed Student t-test was used. Differences were considered significant at P<0.05.

For imaging in these 5 mice, 293T cells stably expressing Firefly luciferase enzyme were used as secondary gene marked cells to report on overall tumor viability when admixed with 293T cells stably expressing split TK plus the interacting proteins FRB and FKBP12 under study. We therefore used Firefly luciferase activity measured by optical CCD camera imaging as an indirect crude measure of the viability and adequacy of cell number of admixed 293T cells acting as the source of the microPET images. Details of optical imaging are as reported previously (Proc. Natl. Acad. Sci. USA 99, 15608-15613 (2002), which is incorporated herein by reference regarding the material related to this discussion). We removed the tumors from the animals after microPET imaging for immunostaining to confirm the expression of split TK protein within the tumors (Supplementary Methods and FIG. 11).

### Supplemental Information for Example 1:

### Supplementary Methods

### Chemicals, Enzymes and Reagents

Restriction and modification enzymes, and T4 DNA ligase were purchased from New England Biolabs (Beverly, Mass.). PCR amplification was performed with TripleMaster Taq DNA polymerase purchased from Brinkmann Eppendorf (Hamburg, Germany). The CheckMate Mammalian two-hybrid kit containing vectors pBIND-Id and pACT-MyoD was purchased from Promega (Madison, Wis.). The plasmids pC₄EN-F1 (expressing FKBP12) and PC₄-RHE (expressing FRB) were obtained from Ariad Pharmaceuticals, Inc. (Cambridge, Mass.), and pCMV-HSV1-sr39tk was a gift from Dr. Margaret Black (Washington State University, Pullman, Wash.). Superfect transfection reagent, plasmid extraction kits, and DNA gel extraction kits were purchased from Qiagen (Valencia, Calif.). Bacterial culture media were purchased from BD Diagnostic Systems (Sparks, Md.). Lipofectamine 2000 transfection reagent, all animal cell culture media, fetal bovine serum, the antibiotics streptomycin and penicillin, and plastic wares for growing cell cultures were purchased from Invitrogen (Carlsbad, Calif.). Rapamycin was purchased from Sigma (St. Louis, Mo.), and [8-³H]Penciclovir was obtained from Moravek Biochemicals (Brea, Calif.).

### Construction of Plasmids

### Expression Vectors to Generate Circularly Permuted TK Variants:

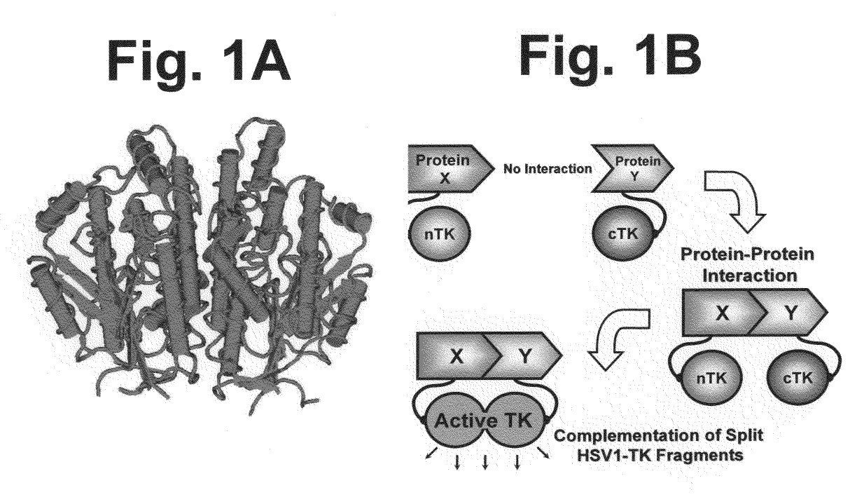

To construct five versions of TK with five different split sites (Supplementary FIG. 1), five N-terminal fragments (nTK) and five C-terminal fragments (cTK) of the HSV1-sr39tk (TK) gene were amplified using the 5′ end forward and 3′ end reverse primers described in Table 1, and pCMV-HSV1-sr39tk as template. For convenient cloning, the forward primer for the upstream gene (cTK) was introduced with an NheI restriction enzyme site and a start codon, both satisfying partial Kozak consensus sequence requirements for expression enhancement. The reverse primer of the upstream gene and the forward primer of the linker gene were introduced with a BamHI restriction enzyme site. The forward primer of the downstream gene (nTK) and the reverse primer of the linker gene were introduced with an EcoRI restriction enzyme site. The reverse primer of the downstream gene was introduced with a XhoI restriction enzyme site and a stop codon. cDNA encoding the flexible linker (GGGGS)₂ was placed between the upstream and downstream genes in each cassette. The digested fragments were cloned into a pcDNA3.1 (+) vector backbone at NheI/XhoI restriction enzyme sites. All cassettes were driven by a CMV promoter. Isolated plasmids were analyzed for the presence of inserts, and positive clones were confirmed for insert presence based on fragment size using restriction endonuclease analysis.

### Expression Vectors for the TK PCA Strategy:

These vectors are shown schematically in FIG. 2a. To construct the vector nTK-Id, nTK and Id were amplified using the 5′ end forward and 3′ end reverse primers described Table 2, and pCMV-HSV1-sr39tk and pBIND-Id as templates, respectively. For convenient cloning, the forward primer for the upstream gene (nTK) was introduced with an NheI restriction enzyme site and a start codon, both satisfying partial Kozak consensus sequence requirements for expression enhancement. The reverse primer of the nTK and the forward primer of the Id gene, with an initial cDNA sequence encoding the flexible linker (GGGGS)₂ (SEQ ID No: 9), were introduced with an EcoRI restriction enzyme site. The reverse primer of Id gene was introduced with a XhoI restriction enzyme site and a stop codon. A similar strategy was used to construct MyoD-cTK, except that the reverse primer of MyoD, with a terminal cDNA sequence encoding the flexible linker (GGGGS)₂, and the forward primer of the cTK gene were introduced with a BamHI restriction enzyme site. The template used was pACT-MyoD. The digested fragments were cloned into a pcDNA3.1(+) vector backbone at NheI/XhoI restriction enzyme sites. Isolated plasmids were analyzed for the presence of inserts, and positive clones were confirmed for insert presence based on fragment size using restriction endonuclease analysis. Similar strategies were used to construct nTK-FRB and FKBP12-cTK. The human FRB fragment was amplified using the forward primer designed with EcoRI and the linker sequence (GGGGS)₂, and the reverse primer designed with XhoI and a stop codon by using the template vector provided by Ariad pharmaceuticals. The amplified fragment was cloned downstream of nTK fragment by using corresponding restriction enzymes. Similarly, the protein fragment FKBP12 was amplified using the forward primer designed with NheI and a start codon, and the reverse primer designed with BamHI by using the template provided by Ariad pharmaceuticals. The digested fragment was cloned upstream of cTK fragment by digesting with corresponding enzymes. Further details of the cloning methods and the primer sequences used are available upon request. Using similar cloning strategies, a further 6 variations on nTK-FRB and FKBP12-cTK were constructed to obtain protein chimeras with different orientations, as described in text.

### Expression Vectors for TK Fragment Point Mutations:

These vectors are shown schematically in FIG. 3a. To generate nTK with the point mutation V119C, site directed mutagenesis (kit obtained from Strategene, La Jolla, Calif.) was performed with the forward primer 5′gtaatgacaagcgcccagataaca (SEQ ID No: 10) and the reverse primer 5′gcacgccgcgtccccggccgatat (SEQ ID No: 11) synthesized at the Stanford Protein and Nucleic Acid Facility. Similarly, to generate cTK with the point mutation R318C, site directed mutagenesis was performed with the forward primer 5′cgtcttggccaaatgtctccgtcccatgc (SEQ ID No: 12) and the reverse primer 5′gcatgggacggagacatttggccaagacg (SEQ ID No: 13). Isolated plasmids were analyzed for the presence of inserts, and positive clones were confirmed for insert presence based on fragment size using restriction endonuclease analysis. The mutants were verified by sequencing.

### Expression Vectors for Preparing Stable 293T Cells:

To prepare stable 293T cells we constructed a single vector system expressing split TK fragments as well as the interacting proteins (FIG. 3c). The above described pcDNA3.1 (puromycin) vector expressing nTK_{(V119C)}-FRB driven by a CMV promoter used for the transient expression study was digested with BgIII restriction enzyme and dephosphorylated. The forward and reverse primers designed with BgIII restriction enzyme sites on either side were used for the PCR amplification of a ubiquitin promoter (pUbi) driving FKBP12-cTK (pUbi-FKBP12-cTK). The BgIII restriction enzyme digested PCR product was ligated into the BgIII digested dephosphorylated pcDNA3.1 (+)-nTK_{(V119C)}-FRB to construct pcDNA-pUbi-FKBP12-cTK-pCMV-nTK_{(V119C)}-FRB. This vector was used for making stable 293T cells. A separate pcDNA3.1 (puromycin) vector containing the Firefly luciferase gene driven by a ubiquitin promoter (pUbi) was used for making a different set of stable 293T cells expressing the Firefly luciferase enzyme.

### Cell Culture

Human embryonic kidney cancer 293T cells (ATCC, Manassas, Va.) were grown in MEM medium supplemented with 10% FBS and 1% penicillin/streptomycin solution.

cell transfection

Transfections were performed in 80% confluent 24 hrs old cultures of 293T cells. For transfection in 12-well culture plates, 250 ng/well of DNA were used when a single construct was used for transfection, giving a total of 500 ng/well when two constructs were combined in a co-transfection. For transfection with pCMV-HSV1-sr39tk alone (as a positive control), 500 ng/well were used. First, single transfections of circularly permuted variants of TK were performed. Next, to assess the PCA strategy using split TK fragments, co-transfection was performed with constructs 4 plus 5 (FIG. 2a), and subsequently with constructs 2 plus 3 (FIG. 2a), also using constructs 6 and 7 (FIG. 2a) as appropriate controls. For assessment of point mutated variants of TK on functioning of the PCA strategy, we co-transfected constructs I and II, I and IV, II and III, II and IV (FIG. 3a). Volumes of Superfect used were as recommended by the manufacturer. For heterodimerization of FRB and FKBP12 in cell culture, 40 nM final concentration of rapamycin were added immediately after transfection. The cells were assayed after 36 hr incubation at 37° C. and in 5% CO₂.

### Rapamycin Escalation Dose Study

Prior to microPET imaging in living mice these cells were also used in a cell-culture rapamycin escalation dose study where net accumulation of probe was measured against increasing rapamycin doses up to 100 nM. Furthermore, since ascomycin (FK506) competes with rapamycin for binding to FKBP12, we studied this competitive binding in these stably transfected 293T cells by adding escalating concentrations (from 0 to 16 μM) of ascomycin along with 40 nM rapamycin 36 h prior to in vitro [8-³H]Penciclovir cell uptake assay.

### Western Immunoblotting

Stably transfected cell samples were also lysed and expression levels of FRB (mTOR) and FKBP12 (either endogenous or in their fusion with split TK fragments) in total lysates were determined by immunoblotting with anti-TK (1:500, Polyclonal, raised in rabbit) for nTK-FRB and FKBP12-cTK, anti-FRB (Cell Signaling) for cellular FRB (mTOR), anti-FKBP12 (Abcam) for cellular FKBP12, and anti-c-tubulin (Sigma) as an internal control for loading.

### Reversibility of the Split TK PCA

We studied the reversibility of our PCA based on split TK using a ligand-reversible dimer of a mutant FKBP12 called F_{M} (F36M) that can be disrupted by FK506¹. We fused F_{M} to split TK fragments and transiently expressed (0.5 μg DNA per well in a 12-well plate) the resulting chimeras, nTK_{(V119c)}-F_{M} and F_{M}-cTK, in a single vector in 293T cells. Twenty-four hours later we added FK506 to these cells and, using an in vitro [8-³H]Penciclovir cell uptake assay, measured either the time- or dose-dependent changes in homodimer dissociation and consequent diminished TK complementation.

### Immunostaining of Tumors for Split TK Expression

We removed the tumors from the animals after microPET imaging, embedded them in OCT in a plastic mold, and froze them in liquid nitrogen. The frozen blocks were sliced using a cryotome and slices were immunostained to confirm the expression of split TK protein within the tumors. Tumor slices of 10 μm from both control (mock transfected 293T cells) and experimental (stable 293T cells with split TK) tumors were fixed on glass slides with acetone for 2 min, after which this was made to evaporate by keeping at room temperature for 10 min. The tissues were blocked by incubating with Tris-Buffered Saline with Tween (TBST) containing 2% bovine serum albumin (BSA) for 60 min at room temperature. The slides were further incubated in TBST with 2% BSA containing anti-TK antibody (1:500, Polyclonal, raised in rabbit) for an additional 60 min. The slides were washed three times (5 min each) with TBST. The slides were then incubated with TBST containing fluorescein-labeled goat anti-rabbit secondary antibody (1:200; Chemicon, Temicula, Calif.) for 60 min at room temperature. The washed slides were mounted with coverslips by using Cytoseal XYL (Microm International, Walldorf, Germany). Fluorescent microscopy (using an excitation filter 365 nm) of these cells was performed using an Axiovert 25 microscope (Carl Zeiss AG, Thornwood, N.Y.) and micrographs were obtained at ×400 magnification using an AxioCam MRc camera (Bernried, Germany).

### Supplementary Discussion

### Circular Permutation Screen for Candidate Complementation Domains of TK

To create a PCA based on TK, the first step was to identify sites where we could disrupt the primary amino acid sequence of TK to separate the enzyme into two complementary protein fragments. Ultimately, the best evidence for whether particular split reporter fragments will work is to determine whether the full polypeptide sequence can be circularly permuted at the proposed points of dissection: this has turned out to be the case for every enzyme that has been used to date in a PCA Being formally equivalent to circularization of a polypeptide chain followed by a cleavage at a site different from the original termini, a circular permutation places charged chain termini at new locations in a protein.

Since many circularly permuted proteins fold into stable, functional conformations, often both in vitro and in vivo, the development of a PCA can be assisted by using circular permutation to identify specific sites within a reporter protein that could be split to create two complementary protein fragments. When constructing circularly permuted variants, particular scrutiny is necessary in considering the distance between the natural N- and C-termini and the relative length of the peptide linker designed to span this distance, so as not to compromise the flexibility at the natural ends of the circular protein. It is surprisingly often observed in nature that the N- and C-termini of folded proteins reside in close proximity; indeed there is a significant preference for them to do so. Williams et al. have indicated that 9 residues seems to represent the minimum number required for a linker that does not introduce conformational strain in an average protein; this number can be derived by representing a random coil polypeptide by the worm-like chain model. Since the distance between the two termini of TK is unknown, we judged that the 15-residue linker used in circular permutation of TK should produce enough flexibility between its two natural termini.

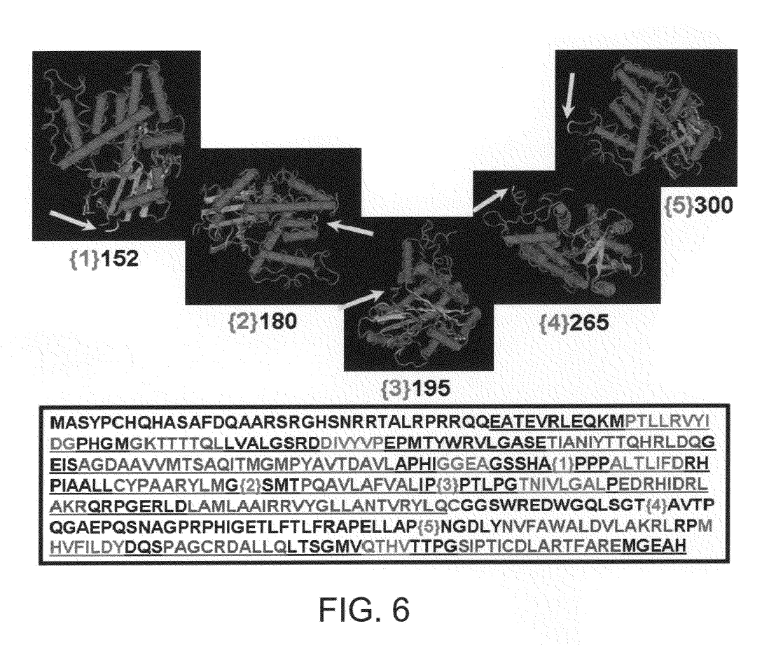

The constituent subunits of TK display the general aβ folding pattern (FIG. 1a). Each aβ structure is made up of 15 α-helices and 7 β-sheets. A 5-stranded parallel β-sheet forms part of the core of the molecule, which contains 5 active sites (Protein Sci. 6, 2097-2106 (1997), which is incorporated herein by reference regarding the material related to this discussion). There are no long cleft-like peptide loop segments in its backbone amenable to single site cleavage, as was the case when splitting Firefly luciferase or GAR transformylase. A full circular permutation analysis (e.g., previously undertaken in E. Coli disulphide oxidoreductase DsbA [189 residues long] (J. Mol. Biol. 286, 1197-1215 (1999), which is incorporated herein by reference regarding the material related to this discussion), and in DHFR [159 residues long] (Nat. Struct. Biol. 7, 580-585 (2000), which is incorporated herein by reference regarding the material related to this discussion)) involving every peptide bond in the TK molecule was deemed too exhaustive and impractical a proposition. Since there are no obvious structural clues from the TK molecule as to where to home in precisely in order to increase the likelihood of a successful cleavage, five potential cleavage sites were simultaneously investigated in a partial circular permutation analysis. None of the five sites chosen for splitting were within regions of periodic secondary structure; all were within disordered loop regions (FIG. 6). Three split sites (between Ala-152 and Pro-153, Gly-180 and Ser-181, and Pro-195 and Pro-196) were within active sites of TK, and another two split sites (between Thr-265 and Ala-266, and Pro-300 and Asn-301) were outside its active sites. Two split sites (between Gly-180 and Ser-181, and Pro-300 and Asn-301) were in regions of the molecule at the dimerization interface with the opposing homodimer. All five split sites were present on the surface of TK (FIG. 6).

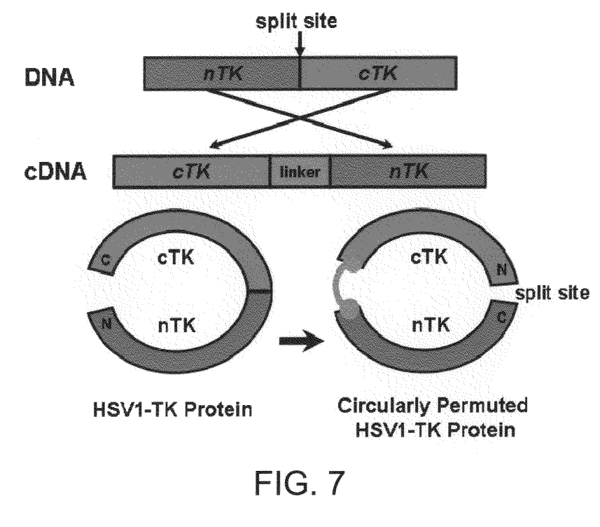

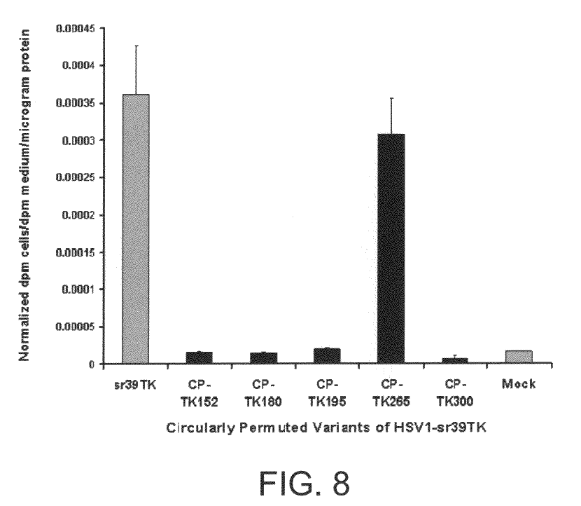

We produced all circular permuted genes using polymerase chain reaction to amplify the desired region of a tethered head to tail dimer gene derived from HSV1-sr39tk (TK), in the form N-[cTK]-C-linker-N-[nTK]-C, where nTK and cTK represent the portions of the HSV1-sr39TK gene proximal and distal to the split site, respectively (FIG. 7). The tethered dimer gene contained a DNA sequence encoding a flexible 15-amino acid [(GGGGS)₃ (SEQ ID No: 15)] linker that connects the natural N-terminus of one copy of nTK to the natural C-terminus of the second copy of cTK. The linker used is of standard composition, made of 3 repeats of a penta-peptide composed of glycine (a neutral/normophobic amino acid with the smallest molecular weight [75 Da] and smallest side chains amongst all amino acids, and serine, also a neutral amino acid with the third smallest molecular weight of 105 Da). We studied the functional effects of the engineered five circularly permuted variants of TK using in vitro TK enzyme uptake assays in 293T cells, and by comparing the properties of these mutated proteins with the original TK. The circularly permuted variant _{CP}TK₂₆₅ (where the split site was between Thr-265 and Ala-266) was the only mutation to retain TK enzymatic activity (85.2%, as compared to full length normal HSV1-sr39TK) (FIG. 8). All other circularly permuted variants (_{CP}TK₁₅₂, _{CP}TK₁₈₀, _{CP}TK₁₉₅, _{CP}TK₃₀₀) were enzymatically inactive.

### Temporal Changes in Complementation of Split TK Fragments

We undertook to combine an analysis of the co-expression of the fusion proteins described above with a temporal study to show how the resulting TK complementation might develop over the first 60 hours following transient transfection in 293T cells. This tactic seemed necessary on account of the findings of Gautier et al. who demonstrated, using homo-FRET anisotropy decay of GFP-tagged TK molecules, that TK homodimerization can only be observed beyond 24 hours of transient transfection in Vero cells. Accordingly, TK would seem to exist as a monomer in the first 24 hours after transient transfection, and in a monomer/dimer equilibrium thereafter. We found the greatest amount of activity of intact TK was at 36 hours after transient transfection (FIG. 9), presumably early during the onset of TK homodimerization. Three other observations were apparent at this time period: the overall degree of restored TK activity was small, there was only a small rise in TK complementation upon adding rapamycin, and TK complementation was also detectable prior to adding rapamycin.

### Rapamycin Dose for Heterodimerization-Induced Complementation of Split TK Fragments

Although low levels of restored enzyme activity were obtained in previous successful PCAs (e.g., 20% restored activity was achieved for humanized Renilla luciferase when used with the FRB/FKBP12 system) we investigated if this low level of TK complementation was possibly due to an inadequate dose of rapamycin incapable of fully mediating heterodimerization of FRB and FKBP12.

We had previously evaluated the optimal concentration of rapamycin for efficient heterodimerization-associated recovery of complemented synthetic Renilla luciferase activity when using the FRB/FKBP12 system in 293T cells (Nat. Struct. Biol. 7, 580-585 (2000), which is incorporated herein by reference regarding the material related to this discussion). The optimal rapamycin dose for routine use was 20-40 nM per well in a 12-well plate. The dose of rapamycin used in the above-described temporal study of heterodimerization-associated recovery of complemented TK activity was this optimal dose of 40 nM per well in a 12-well plate. In a prior study, Remy and Michnick used 20 nM per well when evaluating a DHFR-based PCA system in CHO DUKX-B11 cells (Proc. Natl. Acad. Sci. USA 96, 5394-5399 (1999), which is incorporated herein by reference regarding the material related to this discussion). We therefore compared in a separate study the complemented TK activity upon using 4 nM and 40 nM rapamycin per well in a 12-well plate. The recovered TK activity with the larger rapamycin dose was only slightly higher (FIG. 10). Therefore, the overall relatively low level of complemented TK activity observed with heterodimerization of FRB and FKBP12, using split TK at position 265/266, was not likely due to an inadequate dose of the dimerizer rapamycin. Despite rapamycin being a known cell-cycle inhibitor, 40 nM of rapamycin per well is known to be non-toxic to cells, since the typical EC₅₀ necessary to arrest cell division is about 50 times more than the concentration range used here. Indeed, the peak effects of induced heterodimerization of two proteins using rapamycin are generally seen at recommended concentrations of 50-100 nM.

### Self-Complementation of Split TK Fragments

In a PCA strategy, spontaneous unassisted folding of split reporter fragments would lead to a false-positive reporter signal, a situation that would hopelessly confound attempts at interpreting the presence or absence of a protein-protein interaction under study (Curr. Opin. Struct. Biol. 11, 472-477 (2001), which is incorporated herein by reference regarding the material related to this discussion). In the previous experiment, self-complementation of TK fragments was evident from the presence of restored TK activity upon co-expressing nTK-FRB and FKBP12-cTK in the absence of rapamycin (32.2% of the levels of complementation achieved upon protein-protein interaction, for constructs F+G in (FIG. 2d).

To confirm or refute this finding we performed a separate PCA study using another pair of known interacting proteins, after replacing FRB and FKBP12 with Id and MyoD respectively (FIG. 2a, and FIG. 12). MyoD and Id are members of the basic-helix-loop-helix family of nuclear transcription factors, and are known to strongly interact in vivo (J. Biol. Chem. 272, 19785-19793 (1997), which is incorporated herein by reference regarding the material related to this discussion). We tested the interaction of Id and MyoD upon transient co-expression of the fusion proteins nTK-Id and MyoD-cTK in 293T cells, followed by an in vitro TK enzyme cellular uptake assay to detect dimerization-assisted complementation of the TK fragments. Again, this same orientation of chimeras using Id and MyoD was used previously in PCAs with split Firefly luciferase and Renilla luciferase. We compared this with complementation obtained using the previous chimeras containing FRB and FKBP12, before and after addition of rapamycin. As negative controls we also assayed for TK activity upon single- and co-expression of nTK and cTK fragments without any attached proteins to check whether any measured TK activity might originate in either the N-terminal or C-terminal fragments of TK alone, or perhaps by their spontaneous self-complementation even without attached proteins. The results, shown in FIG. 12, confirmed the findings described above using the FRB/FKBP12/rapamycin system (FIGS. 9, 10, 12). However, no perceptible complementation of TK was seen upon interaction of Id and MyoD. We also recorded no activity from the nTK or the cTK fragments alone, but a significant amount of complemented TK was measured upon co-expression of nTK and cTK, providing further evidence for the likely presence of self-complementation of nTK and cTK obtained through cleavage of TK at position 265/266. This self-complementation was unassisted; it could be demonstrated in the absence as well as in the presence of interacting proteins.

Although self complementing reporter fragments might be useful for assaying of other cellular and subcellular events, as recently published by us using split optical imaging reporters, self complementation is detrimental in the context of measuring protein-protein interactions. This is because when there is restored reporter activity upon an apparent protein-protein interaction it is not possible to ascertain if this is a consequence of the protein-protein interaction driving the complementation of split fragments, or if it follows self-complementation of these fragments, or both.

### Interacting Proteins Sterically Hinder Complementation of Split TK Fragments

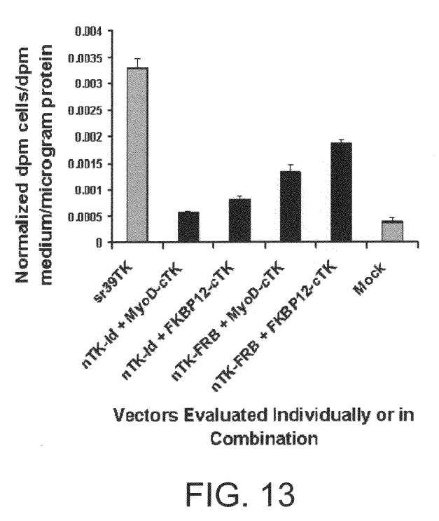

In a successful PCA strategy the efficiencies of complementation between the split reporter fragments must be equivalent once they have been brought together by interactions between alternative interaction partners, i.e. steric hindrance should be avoided. We reasoned that perhaps owing to the larger sizes of Id and MyoD (14 kDa and 35 kDa, respectively) relative to FRB and FKBP12 (10 kDa and 12 kDa, respectively) there might be an element of steric hindrance imposed by the interacting proteins on the split TK fragments in their attempt to complement. We tested the interaction of Id and its usual partner MyoD, as well as FRB and its usual partner FKBP12, upon transient co-expression of the respective fusion proteins in 293T cells, followed by an in vitro TK enzyme cellular uptake assay to detect dimerization-assisted complementation of the TK fragments. We compared this with complementation obtained following deliberate mismatching of interacting partners, such that the nTK-Id chimera was co-expressed with FKBP12-cTK, and nTK-FRB was co-expressed with MyoD-cTK. The results indicated an interesting gradation in the level of complemented TK activity in relation to the size of the protein attached to nTK primarily (the larger the protein, the less enzyme activity obtained), and the size of the protein attached to cTK secondarily (also, the larger the protein, the less enzyme activity obtained) (FIG. 13). These findings indicate that some degree of steric hindrance by these particular interacting proteins might contribute to the overall lower levels than hoped for of complementation of the split TK fragments observed in this study.

We also showed the likelihood that interacting protein partners sterically hinder the complementation of split TK fragments to variable degrees, depending on the size of the interacting proteins. This might account in some measure for why there was a greater extent of TK fragment complementation when using FRB/FKBP12 than Id/MyoD as test proteins. Such a limitation in a PCA strategy may arise especially when interactions between large proteins or giant oligomeric proteins are being investigated. Under such circumstances, steric hindrance by the large proteins can possibly inhibit the interactions between the two separate reporter fragments.

### Reversibility of the Split TK PCA

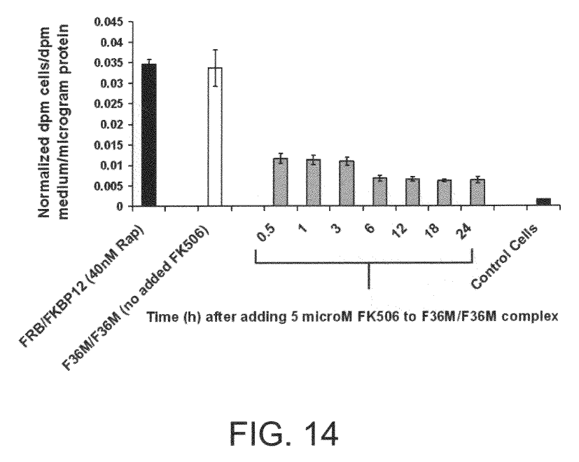

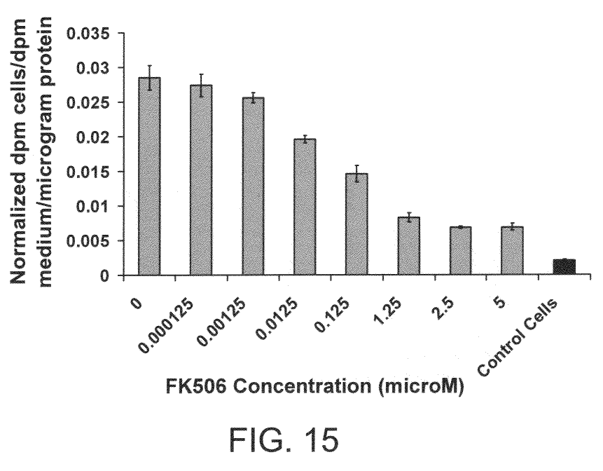

We separately studied the reversibility of our split TK-based PCA using a ligand-reversible dimer of a mutant FKBP12 called F_{M} (F36M) that can be disrupted by FK506¹. We fused F_{M} to split TK fragments and transiently expressed the resulting chimeras, nTK_{(V119C)}-F_{M} and F_{M}-cTK, in a single vector in 293T cells. Twenty-four hours later we added FK506 to these cells and, using an in vitro [8-³H]Penciclovir cell uptake assay, measured either the time- or dose-dependent changes in homodimer dissociation and consequent diminished TK complementation. Five μM FK506 resulted in dissociation of about two-thirds of complexes within 30 min (FIG. 14), and incremental reduction of TK activity was observed with increasing doses of FK506 up to 5 μM (measured after 24 hr of exposure to this ligand) (FIG. 15). This observed reversibility of our PCA confirms that disassembly of the folded TK reporter is possible even after split TK fragments have complemented following a protein-protein interaction.

### Immunostaining of Tumors for Split TK Expression

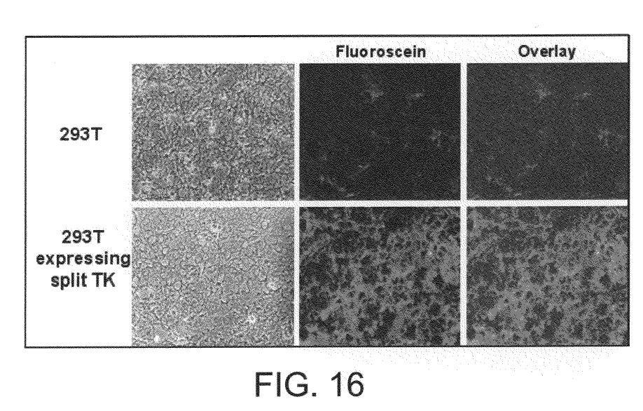

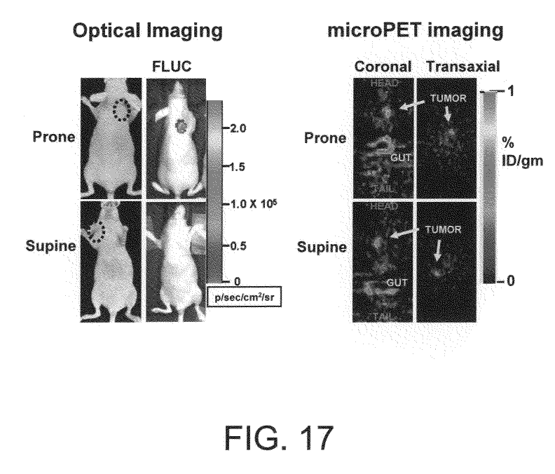

The immunohistochemical analysis of control and experimental tumors showed considerable levels of TK protein expression from the tumors developed from 293T cells stably expressing split TK plus the interacting proteins, and not from the control mock transfected 293T cells (FIG. 16).

TABLE 1

Example 1
Nucleotide Sequence and the Positions of PCR
Primers with Linkers Used for Constructing
the Different Expression Vectors in the
Study of Circularly Permuted Variants of TK
(SEQ ID Nos: 14-27)

| | | |
|---|---|---|
| PRIMER NAME | PRIMER SEQUENCE (5′→3′) | POSITION |

| | | |
|---|---|---|
| Forward | atat gaattc gct tcg tac ccc | 1-8 |
| nTK (14) | tgc cat caa cac | |
| Reverse | atat ggatcc gtt agc ctc ccc | 376-369 |
| cTK (15) | cat ctc ccg ggc | |
| Reverse | atat ctcgag tca ggc atg tga | 152-145 |
| nTK₁₅₂ (16) | gct ccc agc ctc ccc | |
| Forward | atat gctagc atg ccg ccc ccg | 153-158 |
| cTK₁₅₂ (17) | gcc ctc acc | |
| Reverse | atat ctcgag tca gcc cat aag | 180-172 |
| nTK₁₈₀ (18) | gta tcg cgc ggc cgg | |
| Forward | atat gctagc atg agc atg acc | 181-188 |
| cTK₁₈₀ (19) | ccc cag gcc gtg ctg | |
| Reverse | atat ctcgag tca cgg gat gag | 195-188 |
| nTK₁₉₅ (20) | ggc cac gaa cgc cag | |
| Forward | atat gctagc atg ccg acc ttg | 196-203 |
| cTK₁₉₅ (21) | ccc ggc aca aac atc | |
| Reverse | atat ctcgag tca cgt ccc cga | 265-258 |
| nTK₂₆₅ (22) | aag ctg tcc cca atc | |
| Forward | atat gctagc atg gcc gtg ccg | 266-273 |
| cTK₂₆₅ (23) | ccc cag ggt gcc gag | |
| Reverse | atat ctcgag tca aac tcg ggg | 300-293 |
| nTK₃₀₀ (24) | gcc cga aac agg gta | |
| Forward | atat gctagc atg gct ggc ccc | 301-308 |
| cTK₃₀₀ (25) | caa cgg cga cct gta | |
| Forward | gatcc ggt ggc gga ggg agc | |
| Linker (26) | ggt ggc gga ggg agc ggt ggc | |
| | gga ggg agc g | |
| Reverse | aattc gct ccc tcc gcc acc | |
| Linker (27) | gct ccc tcc gcc acc gct ccc | |
| | tcc gcc acc g | |

*Bold letters are regions of the restriction enzyme recognition site

TABLE 2

Example 1
Nucleotide Sequence of PCR Primers with
Linkers Used for Constructing the Different
Expression Vectors in the Study of the TK
PCA Strategy (with TK split at site
265/266) (SEQ ID Nos: 28-35)

| | |
|---|---|
| PRIMER NAME | PRIMER SEQUENCE (5′→3′) |

| | |
|---|---|
| Forward | atat gctagc atg gct tcg tac ccc tgc cat |
| nTK (28) | caa |
| Reverse | atat gaattc cgt ccc cga aag ctg tcc cca |
| nTK (29) | atc |
| Forward | atat gaattc ggt ggc gga ggg agc ggt ggc |
| Id (30) | gga ggg agc cat aaa ttc cca ctt ggt ctg |
| Reverse | atat ctcgag att aac cct cac taa agg |
| Id (31) | |
| Forward | atat gctagc atg ccg gag tgg cag aaa gtt |
| MyoD (32) | aag acg |
| Reverse | atat ggatcc gct ccc tcc gcc acc gct ccc |
| MyoD (33) | tcc gcc acc ccg aat tcg agc tcg ccc ggg |
| Forward | atat ggatcc tgc gtg ccg ccc cag ggt gcc |
| cTK (34) | gag ccc |
| Reverse | atat ctcgag tca gtt agc ctc ccc cat ctc |
| cTK (35) | ccg |

*Bold letters are regions of the restriction enzyme recognition site

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, +5%, +6%, +7%, +8%, +9%, or +10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

TK protein
SEQ ID No: 1
Amino acid sequence:
masypchqha safdqaarsr ghsnrrtalr prrqqeatev
rleqkmptll rvyidgphgmgkttttqllv algsrddivy
vpepmtywrv lgasetiani yttqhrldqg eisagdaavvmtsaqit
mgm pyavtdavla phiggeagss happpaltli fdrhpiaall
cypaarylmgsmtpqavlaf valipptlpg tnivlgalpe
drhidrlakr qrpgerldla mlaairrvygllantvrylq
cggswredwg qlsgtavtpq gaepqsnagp rphigetlft lfrape
llapngdlynvfaw aldvlakrlr pmhvfildyd qspagcrdal
lqltsgmvqt hvttpgsipticdlartfar emgeah
TK nucleotide sequence
SEQ ID No: 2
Nucleotide sequence:
atggcttcgt acccctgcca tcaacacgcg tctgcgttcg
accaggctgc gcgttctcgcggccatagca accgacgtac
ggcgttgcgc cctcgccggc agcaagaagc cacggaagtccgcctgg
agc agaaaatgcc cacgctactg cgggtttata tagacggtcc
tcacgggatggggaaaacca ccaccacgca actgctggtg
gccctgggtt cgcgcgacga tatcgtctacgtacccgagc
cgatgactta ctggcaggtg ctgggggctt ccgagacaat cgcgaa
catctacaccacac aacaccgcct cgaccagggt gagatatcgg
ccggggacgc ggcggtggtaatgacaagcg cccagataac
aatgggcatg ccttatgccg tgaccgacgc cgttctggctcctcata
tcg ggggggaggc tgggagctca catgccccgc ccccggccct
caccctcatcttcgaccgcc atcccatcgc cgccctcctg
tgctacccgg ccgcgcgata ccttatgggcagcatgaccc
cccaggccgt gctggcgttc gtggccctca tcccgccgac cttgcc
cggcacaaacatcg tgttgggggc ccttccggag gacagacaca
tcgaccgcct ggccaaacgccagcgccccg gcgagcggct
tgacctggct atgctggccg cgattcgccg cgtttacgggctgcttg
cca atacggtgcg gtatctgcag ggcggcgggt cgtggcggga gg
attggggacagctttcgg ggacggcogt gccgccccag
ggtgccgagc cccagagcaa cgcgggcccacgaccccata
tcggggacac gttatttacc ctgtttcggg cccccgagtt gctggc
ccccaacggcgacc tgtataacgt gtttgcctgg gccttggacg
tcttggccaa acgcctccgtcccatgcacg tctttatcct
ggattacgac caatcgcccg ccggctgccg ggacgccctgctgcaac
tta cctccgggat ggtccagacc cacgtcacca ccccaggctc ca
taccgacgatctgcgacc tggcgcgcac gtttgcccgg
gagatggggg aggctaactg a

We claim the following:

1. A split protein herpes simplex virus type 1 thymidine kinase (TK) system, comprising:
a first TK protein including a first TK self complementing fragment, wherein the first TK self complementing fragment comprises a first portion of a TK protein, and
a second TK protein including a second TK self complementing fragment, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein.

2. The split protein system of claim 1, wherein the first TK protein includes a first target protein and the first TK self complementing fragment.

3. The split protein system of claim 1, wherein the first TK protein includes a first target protein and the first TK self complementing fragment, and wherein the second TK protein includes a second target protein and the second TK self complementing fragment.

4. The split protein system of claim 1, wherein the first TK self complementing fragment is selected from one of the following: an expressed protein from a N-terminal fragment of a TK gene having SEQ ID No. 2, or an expressed protein from a C-terminal fragment of the TK gene having SEQ ID No. 2; wherein the second TK self complementing fragment is selected from one of the following: an expressed protein from a N-terminal fragment of a TK gene having SEQ ID No. 2, or an expressed protein from a C-terminal fragment of the TK gene having SEQ ID No. 2; and wherein the first TK self complementing fragment and the second TK self complementing fragment are not the same.

5. The split protein system of claim 1, wherein the first TK self complementing fragment is selected from one of the following: a N-terminal fragment of a TK protein having SEQ ID No. 1, or a C fragment of the TK gene, SEQ ID No. 2; wherein the second TK self complementing fragment is selected from one of the following: a N fragment of a TK gene, SEQ ID No. 2, or a C fragment of the TK gene, SEQ ID No. 2; and wherein the first TK self complementing fragment and the second TK self complementing fragment are not the same.

6. The split protein system of claim 5, wherein the N-terminal fragment is selected from: a fragment having amino acids 1-265 of SEQ ID NO: 1; and wherein the C-terminal fragment is selected from: a fragment having amino acids 266-376 of SEQ ID NO: 1.

7. The split protein system of claim 5, wherein the N-terminal fragment is selected from: a fragment having amino acids 1-265 of SEQ ID NO: 1 having a point mutation of V119C; and wherein the C-terminal fragment is selected from: a fragment having amino acids 266-376 of SEQ ID NO: 1.

8. A method of producing the split protein system, comprising:
providing a first vector that includes a first polynucleotide that encodes a first herpes simplex virus type 1 thymidine kinase (TK) protein including a first TK self complementing fragment, wherein the first TK self complementing fragment comprises a first portion of a TK protein;
expressing the first polynucleotide to produce the first TK protein in a first system;
providing a second vector that includes a second polynucleotide sequence that encodes a second TK protein including a second TK self complementing fragment, wherein the second TK self complementing fragment comprises a second portion of the TK protein that is complementary with the first self complementing fragment, wherein the first self complementing fragment and the second self complementing fragment are not active individually, and wherein the first TK self complementing fragment and the second TK self complementing fragment spontaneously self complement to substantially form an active TK protein; and
expressing the second polynucleotide to produce the second TK protein in a second system.

9. A method of detecting protein-protein interaction, comprising:
providing a first vector that includes a first polynucleotide that encodes a first herpes simplex virus type 1 thymidine kinase (TK) protein including a first TK self complementing fragment and a first target protein, wherein the first TK self complementing fragment comprises a first portion of a TK protein;
expressing the first polynucleotide to produce the first TK protein;
providing a second vector that includes a second polynucleotide sequence that encodes a second TK protein including a second TK self complementing fragment and a second target protein, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein;
expressing the second polynucleotide to produce the second TK protein;
providing a labeled TK substrate, wherein the label of the labeled TK substrate being able to generate a signal; and
generating a signal from the label if the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

10. The method of claim 9, further comprising detecting the signal, wherein detection of the signal indicates that the first protein and the second protein interacted with one another.

11. The method of claim 10, wherein a location of the interaction of the first protein and the second protein is detected by detecting the signal.

12. The method of claim 9, wherein the first protein and the second protein interact in a cell, wherein the labeled TK substrate is adapted to enter the cell, wherein the modified labeled TK substrate is adapted to be retained in the cell so that the modified labeled TK substrate accumulates in the cell.

13. The method of claim 9, wherein the first TK self complementing fragment is selected from one of the following: a N-terminal fragment of a TK protein having SEQ ID No. 1, or a C-terminal fragment of the TK gene, SEQ ID No. 2; wherein the second TK self complementing fragment is selected from one of the following: a N fragment of a TK gene, SEQ ID No. 2, or a C-terminal fragment of the TK gene, SEQ ID No. 2; and wherein the first TK self complementing fragment and the second TK self complementing fragment are not the same.

14. The method of claim 9, wherein the N-terminal fragment is selected from: a fragment having amino acids 1-265 of SEQ ID NO: 1; and wherein the C-terminal fragment is selected from: a fragment having amino acids 266-376 of SEQ ID NO: 1.

15. The method of claim 9, wherein the N-terminal fragment is selected from: a fragment having amino acids 1-265 of SEQ ID NO: 1 having a point mutation of V119C; and wherein the C-terminal fragment is selected from: a fragment having amino acids 266-376 of SEQ ID NO: 1.

16. A method of detecting protein-protein interaction, comprising:
providing a first herpes simplex virus type 1 thymidine kinase (TK) protein, wherein the first TK protein includes a first TK self complementing fragment and a first target protein, wherein the first TK self complementing fragment comprises a first portion of a TK protein;
providing a second TK protein, wherein the second TK protein includes a second TK self complementing fragment and a second target protein, wherein the second TK self complementing fragment comprises a second portion of the TK protein that is complementary with the first self complementing fragment, wherein the first TK self complementing fragment and the second TK self complementing fragment are not active individually, and wherein the first TK self complementing fragment and the second TK self complementing fragment spontaneously self complement to substantially form an active TK protein;
providing a labeled TK substrate, wherein the label of the labeled TK substrate being able to generate a signal; and
generating a signal from the label if the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

17. The method of claim 16, further comprising detecting the signal, wherein detection of the signal indicates that the first protein and the second protein interacted with one another.

18. The method of claim 16, wherein a location of the interaction of the first protein and the second protein is detected by detecting the signal.

19. The method of claim 16, wherein the first protein and the second protein interact in a cell, wherein the labeled TK substrate is adapted to enter the cell, wherein the modified labeled TK substrate is adapted to be retained in the cell so that the modified labeled TK substrate accumulates in the cell.

20. A method of detecting protein-protein interaction, including:
providing a first herpes simplex virus type 1 thymidine kinase (TK) protein, wherein the first TK protein includes a first TK self complementing fragment and a first target protein, wherein the first TK self complementing fragment comprises a first portion of a TK protein;
exposing the first TK protein to a cell, wherein the cell comprises a second TK protein, wherein the second TK protein includes a second TK self complementing fragment and a second target protein, wherein the second TK self complementing fragment comprises a second portion of the TK protein that is complementary with the first self complementing fragment, wherein the first self complementing fragment and the second self complementing fragment are not active individually, and wherein the first TK self complementing fragment and the second TK self complementing fragment spontaneously self complement to substantially form an active TK protein;
introducing a labeled TK substrate to the cell, wherein the label of the labeled TK substrate being able to generate a signal; and
generating a signal from the label if the first target protein enter the cell and the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

21. The method of claim 20, wherein the labeled TK substrate is adapted to enter the cell, wherein the modified labeled TK substrate is adapted to be retained in the cell so that the modified labeled TK substrate accumulates in the cell.

22. A method of cellular localization of proteins, including:
providing a first herpes simplex virus type 1 thymidine kinase (TK) protein, wherein the first TK protein includes a first TK self complementing fragment and a first target protein, wherein the first TK self complementing fragment comprises a first portion of a TK protein;
exposing the first protein to a cell, wherein a compartment of the cell comprises a second TK protein, wherein the second TK protein includes a second TK self complementing fragment and a second target protein, wherein the second TK self complementing fragment comprises a second portion of the TK protein that is complementary with the first TK self complementing fragment, wherein the first TK self complementing fragment and the second TK self complementing fragment are not active individually, and wherein the first TK self complementing fragment and the second TK self complementing fragment spontaneously self complement to substantially form an active TK protein;
introducing a labeled TK substrate to the cell, wherein the label of the labeled TK substrate being able to generate a signal; and
generating a signal from the label if the first target protein enter the compartment of the cell and the first target protein and the second target protein interact, wherein if the first target protein and the second target protein interact, the first TK self complementing fragment and the second TK self complementing fragment spontaneously self complement to substantially form the active TK protein, and wherein the active TK protein interacts with a labeled TK substrate to form a modified labeled TK substrate.

23. A fusion protein, comprising:
a TK protein including a TK self complementing fragment and a target, wherein the TK self complementing fragment comprises a first portion of a TK protein.

24. The fusion of claim 23, wherein the TK self complementing fragment is selected from one of the following: a N-terminal fragment of a TK protein having SEQ ID No. 1, or a C-terminal fragment of the TK gene, SEQ ID No. 2.

25. The fusion of claim 23, wherein the N-terminal fragment is selected from: a fragment having amino acids 1-265 of SEQ ID NO: 1; and wherein the C-terminal fragment is selected from: a fragment having amino acids 266-376 of SEQ ID NO: 1.

26. The fusion of claim 23, wherein the N-terminal fragment is selected from: a fragment having amino acids 1-265 of SEQ ID NO: 1 having a point mutation of V119C; and wherein the C-terminal fragment is selected from: a fragment having amino acids 266-376 of SEQ ID NO: 1.
